(19) United States
(12) Patent Application Publication
(10) Pub. No.: US 2023/0285755 A1
(43) Pub. Date: Sep. 14, 2023

(54) SYSTEMS AND METHODS FOR PREDICTION AND DESIGN OF NEURAL STIMULATION

(71) Applicant: Duke University, Durham, NC (US)

(72) Inventors: Warren Grill, Durham, NC (US); Nicole Pelot, Durham, NC (US); Minhaj Hussain, Durham, NC (US)

(21) Appl. No.: 18/182,018

(22) Filed: Mar. 10, 2023

Related U.S. Application Data
(60) Provisional application No. 63/318,491, filed on Mar. 10, 2022.

(51) Int. Cl. A61N 1/36; G06N 3/0442; G06N 3/084

(52) U.S. Cl. CPC A61N 1/36146; G06N 3/0442; G06N 3/084

(57) ABSTRACT

The present disclosure describes novel systems and methods to estimate the response of neurons to electrical stimulation and to determine the optimal electrode geometry and parameters of stimulation to activate or block specific targeted groups of neurons without activation or block of non-targeted groups of neurons.

### GOVERNMENT FUNDING

This invention was made with Government support under Federal Grant No. OT2-OD025340 awarded by the National Institutes of Health. The Federal Government has certain rights to the invention.

### RELATED APPLICATIONS

This application claims priority to and the benefit of U.S. Provisional Pat. Application No. 63/318,491 filed Mar. 10, 2022, which is incorporated herein by reference in its entirety for all purposes.

### BACKGROUND

Bioelectronic therapy uses implantable medical devices to deliver electrical stimulation to patients to treat a variety of conditions such as heart failure, depression, epilepsy, movement disorders, and chronic pain. Stimulation is delivered via one or more leads with electrode contacts located in proximity to some targeted neural tissue, e.g., the vagus nerve, pelvic nerves, spinal cord, or brain.

The continued advance of bioelectronic therapies is limited by inadequate activation of targeted nerve fibers and by co-activation of non-targeted nerve fibers. More fundamentally, the relationship between applied stimuli and the complement of nerve fibers that are activated or blocked or have a specific subthreshold response, how this relationship varies across individuals and species, and how this relationship can be controlled remain largely unknown.

One of the substantial challenges to addressing these problems is the exceedingly large computational costs of calculating the response of populations of highly nonlinear neurons to different parameters of stimulation. This challenge is compounded when considering the very large number of iterations required for model-based optimization. Hence there is an ongoing need for improved modeling and design of neural stimulation.

### SUMMARY

The Summary is provided to introduce a selection of concepts that are further described below in the Detailed Description. This Summary is not intended to identify key or essential features of the claimed subject matter, nor is it intended to be used as an aid in limiting the scope of the claimed subject matter.

Embodiments of the present disclosure include a method for estimation of a neuronal response. The method comprises inputting a sequence of numerical input vectors corresponding to an electric field over a period of neuronal stimulation into a surrogate model of neuronal stimulation. The surrogate model of neuronal stimulation outputs a sequence of numerical output vectors corresponding to one or more changes in neuronal response during the stimulation period.

In some embodiments, the sequence of numerical input vectors comprise a spatiotemporal distribution of extracellular potentials measured, calculated, or estimated for the period of neuronal stimulation.

In some embodiments, the one or more changes in neuronal response corresponding to the sequence of numerical output vectors comprise at least one of: transmembrane voltage, total transmembrane current, transmembrane current for one or more ions, conduction state of an ion channel, detection of one or more action potentials, a probability of a dynamic event, gating variable values, or any combination thereof.

In some embodiments, the surrogate model comprises a machine learning component and/or an artificial intelligence component comprising at least one of: recurrent neural network, convolutional neural network, fully connected neural network, decision tree, random forest, support vector machine, linear regression, logistic regression, nearest neighbors, or any combination thereof.

In some embodiments, the method further includes training the surrogate model of neuronal stimulation with data from simulation of a biophysical model or a simplified model.

In some embodiments, the sequence of numerical input vectors is from a model of an anatomically realistic nerve morphology with an electrode.

In some embodiments, the surrogate model is a classification surrogate that determines a probability that an action potential has occurred.

In some embodiments, the surrogate model determines a strength-duration relationship, determines a relationship between activation thresholds and neuron size, determines a relationship between block thresholds and neuron size, determines subthreshold response, identifies unidirectional propagation, identifies bidirectional propagation, determines propagation speed, determines the number of evoked action potentials, determines the time of action potential initiation, determines the location of action potential initiation, determines a gating variable, captures interaction between propagating action potentials, captures interaction between a subthreshold change in membrane state and a propagating action potential, captures interaction between changes in membrane state and responses to stimulation, predicts a state of conduction block, or any combination thereof.

In some embodiments, the surrogate model is a cable surrogate that includes nodal compartments of a complete neuron model.

In some embodiments, the surrogate model is fully differentiable with respect to all parameters.

Embodiments of the present disclosure include a method of administering neuromodulation therapy to a subject. The method includes programming a pulse generator to deliver a pattern of electrical stimulation identified using the method for estimation of a neuronal response disclosed herein; and delivering the pattern of electrical simulation to the subject to generate a response in at least one target neuron.

Embodiments of the present disclosure include a method for optimizing neuronal response. The method comprises inputting a set of criteria corresponding to a desired neuronal response into an inverted model of neuronal response. The inverted model of neuronal response outputs one or more stimulation criteria capable of producing the desired neuronal response.

In some embodiments, the set of criteria corresponding to the desired neuronal response comprises identifying one or more target neuron(s) for activation or conduction block. In some embodiments, the set of criteria corresponding to the desired neuronal response comprises identifying one or more target neuron(s) having a specific subthreshold response.

In some embodiments, the one or more stimulation criteria capable of producing the desired neuronal response is optimized with a gradient based optimization.

In some embodiments, the one or more stimulation criteria capable of producing the desired neuronal response is optimized with a global optimization algorithm comprising at least one of: a differential evolution algorithm, a genetic algorithm, a particle swarm algorithm, a simulated annealing algorithm, an ant colony algorithm, an estimation of distribution algorithm, a simplex search algorithm, a coordinate descent algorithm, a random search algorithm, a grid search algorithm, a spiral optimization algorithm, a stochastic diffusion search algorithm, or any combination thereof; or optimized with a recommender system with a machine learning component and/or an artificial intelligence component comprising at least one of: recurrent neural network, convolutional neural network, fully connected neural network, decision tree, random forest, support vector machine, linear regression, logistic regression, nearest neighbors, or any combination thereof.

In some embodiments, the one or more stimulation criteria capable of producing the desired neuronal response is optimized for at least one of: minimizing energy required for activation or block or a subthreshold response, minimizing power required for activation or block or a subthreshold response, minimizing charge imbalance in the optimized waveform(s), minimizing onset response produced when the optimized waveform(s) are turned on, maximizing degree of conduction block, minimizing voltage required for activation or block or a subthreshold response with the optimized waveform(s), minimizing current required for activation or block or a subthreshold response with the optimized waveform(s), minimizing charge required for activation or block with the optimized waveform(s), maximizing therapeutic benefit produced by application of the optimized waveform(s), minimizing adverse effects produced by application of the optimized waveform(s), maximizing selectivity of activation or block between neuron types by application of the optimized waveform(s), maximizing selectivity of activation or block between neuron diameters by application of the optimized waveform(s), maximizing selectivity of activation or block between neuron locations by application of the optimized waveform(s), measures related to the complexity of the electrode geometry or stimulation waveform shape, and any combinations thereof.

In some embodiments, the one or more stimulation criteria comprise an electrode geometric parameter comprising at least one of: inter-contact spacing of contacts, number of contacts, shape of contacts, size of contacts, shape of insulation, size of insulation, placement of contacts relative to the insulation, and any combination thereof.

In some embodiments, the one or more stimulation criteria comprise an electrode placement parameter comprising at least one of: electrode implantation depth, electrode displacement from an anatomical landmark, rotation of electrode, and any combination thereof.

In some embodiments, the one or more stimulation criteria comprise a stimulation parameter comprising at least one of: amplitude, pulse width, frequency, waveform shape, delays, and any combination thereof.

Embodiments of the present disclosure include a method of administering neuromodulation therapy to a subject. The method comprises programming a pulse generator to deliver a pattern of electrical stimulation identified using the method for optimizing neuronal response disclosed herein; and delivering the pattern of electrical simulation to the subject to generate a response in at least one target neuron.

Embodiments of the present disclosure also include a method for estimation of one or more stimulation criteria. In accordance with these embodiments, the method includes inputting a sequence of numerical input vectors corresponding to a desired neuronal response to a surrogate model of neuronal stimulation. In some embodiments, the surrogate model outputs a sequence of numerical output vectors corresponding to one or more stimulation criteria capable of producing the desired neuronal response.

In some embodiments, the set of criteria corresponding to the desired neuronal response comprises identifying one or more target neuron(s) for activation or conduction block. In some embodiments, the set of criteria corresponding to the desired neuronal response comprises identifying one or more target neuron(s) having a specific subthreshold response.

In some embodiments, the one or more stimulation criteria capable of producing the desired neuronal response is optimized with a gradient based optimization.

In some embodiments, the one or more stimulation criteria capable of producing the desired neuronal response is optimized with a global optimization algorithm comprising at least one of: a differential evolution algorithm, a genetic algorithm, a particle swarm algorithm, a simulated annealing algorithm, an ant colony algorithm, an estimation of distribution algorithm, a simplex search algorithm, a coordinate descent algorithm, a random search algorithm, a grid search algorithm, a spiral optimization algorithm, a stochastic diffusion search algorithm, or any combination thereof; or optimized with a recommender system with a machine learning component and/or an artificial intelligence component comprising at least one of: recurrent neural network, convolutional neural network, fully connected neural network, decision tree, random forest, support vector machine, linear regression, logistic regression, nearest neighbors, or any combination thereof.

In some embodiments, the one or more stimulation criteria capable of producing the desired neuronal response is optimized for at least one of: minimizing energy required for activation or block or a subthreshold response, minimizing power required for activation or block or a subthreshold response, minimizing charge imbalance in the optimized waveform(s), minimizing onset response produced when the optimized waveform(s) are turned on, maximizing degree of conduction block, minimizing voltage required for activation or block or a subthreshold response with the optimized waveform(s), minimizing current required for activation or block or a subthreshold response with the optimized waveform(s), minimizing charge required for activation or block with the optimized waveform(s), maximizing therapeutic benefit produced by application of the optimized waveform(s), minimizing adverse effects produced by application of the optimized waveform(s), maximizing selectivity of activation or block between neuron types by application of the optimized waveform(s), maximizing selectivity of activation or block between neuron diameters by application of the optimized waveform(s), maximizing selectivity of activation or block between neuron locations by application of the optimized waveform(s), measures related to the complexity of the electrode geometry or stimulation waveform shape, and any combinations thereof.

In some embodiments, the one or more stimulation criteria comprise an electrode geometric parameter comprising at least one of: inter-contact spacing of contacts, number of contacts, shape of contacts, size of contacts, shape of insulation, size of insulation, placement of contacts relative to the insulation, and any combination thereof.

In some embodiments, the one or more stimulation criteria comprise an electrode placement parameter comprising at least one of: electrode implantation depth, electrode displacement from an anatomical landmark, rotation of electrode, and any combination thereof.

In some embodiments, the one or more stimulation criteria comprise a stimulation parameter comprising at least one of: amplitude, pulse width, frequency, waveform shape, delays, and any combination thereof.

### BRIEF DESCRIPTION OF THE DRAWINGS

The accompanying figures are provided by way of illustration and not by way of limitation.

FIG. 1 is a diagram illustrating a system overview for developing surrogate models and performing geometry and/or stimulus parameter optimization: Block 1 illustrates surrogate computational models of a neuron’s biophysical response to extracellular stimulation; Block 2 illustrates optimization of electrode geometry and/or stimulation parameters; and Block 3 illustrates recommender networks to generate model-specific stimulation parameter values to achieve selective activation or blocking of user-defined targets.

FIG. 2 is a schematic of an example implementation of a classification-based surrogate model of a neuron to predict the probability of an action potential occurring due to extracellular electrical stimulation. Panel A: An example of a sequence of extracellular potential distributions. Highlighted in cyan are two arbitrary timesteps between the start and end of stimulation (t1, t2) and highlighted in red is the last non-zero stimulation time-step (1). Panel B: The Gated Recurrent Unit (GRU) unrolled in time illustrating the three timesteps highlighted in panel B (other timesteps are omitted). Illustrated also is the latent state vector h, representing the inactivation variable of the voltage gated sodium channel. Panel C: The final decision (probability of an action potential) is generated via a single fully connected layer with a sigmoid activation operating on the h state vector corresponding to the last relevant GRU state (corresponding to timestep 1), representing the confidence of the model that the given extracellular potential sequence would elicit a propagating action potential. Panel D: Mathematical description of a single GRU cell.

FIG. 3 is an example schematic recurrent neural network architecture for learning neuron responses to extracellular electric fields that vary in space and time. ‘Field t1’ and ‘Field t2’ illustrate example potential distributions along the axon at adjacent timepoints t1 and t2 respectively. The axon states (some subset of dynamic variables describing the biophysical model) at t1 and t2 are computed by forward propagation through a series (1 though L) neural network layers (NN) that each individually maintains an internal state carried forward from the previous timestep. The implementation uses a version of the LSTM network for each layer (NN), however other network architectures (such as Elman network, GRU) may be substituted.

FIG. 4 illustrates strength-duration (SD) curves for a surrogate model (blue traces on right) and a biophysical model (orange traces on right) for indicated source-fascicle pairs; note that the blue traces are not clearly visible because they are under the orange traces. SD-curves for monophasic pulses are in the top panel and biphasic pulses are in the bottom panel. Blue traces are obscured because the predictions agree very well. Depicted on the left is the cross section of pig vagus nerve used to generate the field model. The outermost circle is the nerve boundary and interior traces are fascicles (i.e., bundles of axons). The 6 contacts of the cuff electrode are shown around the nerve boundary, numbered 1 to 6 (only the trace of the contact surface is shown). Each number refers to a single source-axon pair, and each numbered strength-duration relationship is for a single 5.7 µm MRG axon model placed at the center of the numbered fascicle being stimulated using the numbered electrode contact.

FIG. 5 is an illustration of interaction between propagating action potentials, whereby action potentials initiated at different locations along an axon meet and mutually annihilate. ‘m’ is the m gating variable (of the fast sodium channel in the MRG axon model, bounded between 0 and 1) and ‘t’ is time.

FIG. 6 is an illustration of unidirectional action potential propagation. Illustrated is a comparison of the m gating variable (‘m’) for a biophysical (MRG) model axon and surrogate model axon (recurrent network predicting m, p, h, and s gating variables of MRG axon) in the same location within a fascicle near the stimulating electrode. The electrode is delivering a -3 mA monophasic pulse. Both models exhibit unidirectional action potential propagation, where an action potential is initiated beneath the stimulating electrode but only propagates in one direction along the axon.

FIG. 7 is an illustration of onset response and inactivation due to a high frequency signal. Illustrated is a comparison of the m gating variable (‘m’) for a biophysical (MRG) model axon and surrogate model axon (recurrent network predicting m, p, h, and s gating variables of MRG axon) in the same location within a fascicle near the stimulating electrode. The electrode is delivering a 3 mA 20 kHz sinusoid. Both models exhibit initial strong excitation, followed by inactivation, where the m gating parameter remains high beneath the stimulating electrode although no more action potentials are generated.

FIG. 8 is a graph of average calculation times for constructing strength-duration curves as illustrated in FIG. 4 using the biophysical model (NEURON) vs. surrogate gating variable model (NN). The biophysical model (running single threaded on CPU) takes approximately 120 s and the surrogate model takes approximately 8 s (running on GPU).

FIG. 9 is an example scheme of a gradient-free optimization for parameter selection. Convergence for a population-based optimization approach may, for example, be defined in terms of a pre-specified minimum standard-deviation for the losses of a population of candidate solutions.

FIG. 10 illustrates a pig cervical vagus nerve model and optimization results. Left: Histology with motor/sensory distinction. Middle: Cross section of the modeled nerve and six electrode contacts. Blue fascicles are the targets. Red dots indicate active axons as evaluated in NEURON. Right: Stimulus waveforms for each of the six electrode contacts as determined by the differential evolution (DE) optimization. 100% of target fascicles were activated with 0% off-target fascicles activated.

FIG. 11 illustrates a pig cervical vagus nerve model and optimization results. Left: Histology with motor/sensory distinction. Middle: Cross section of the modeled nerve and six electrode contacts. Blue fascicles are the targets. Red dots indicate active axons as evaluated in NEURON. Right: Stimulus waveforms for each of the six electrode contacts as determined by the DE optimization. 95.5% of target fascicles were activated with 9.1% off-target fascicles activated.

FIG. 12 illustrates a pig cervical vagus nerve model and optimization results. Left: Histology with motor/sensory distinction. Middle: Cross section of the modeled nerve and six electrode contacts. Blue fascicles are the targets. Red dots indicate active axons as evaluated in NEURON. Right: Stimulus waveforms for each of the six electrode contacts as determined by the DE optimization. 92% of target fascicles were activated with 4% off-target fascicles activated.

FIG. 13 illustrates a human cervical vagus nerve and optimization results. Left: Cross section of the modeled nerve and six electrode contacts. Blue fascicles are the targets. Red dots indicate active axons as evaluated in NEURON. Right: Stimulus waveforms for each of the six electrode contacts as determined by the DE optimization. 100% of target fascicles were activated with 0% off-target fascicles activated.

FIG. 14 illustrates a human cervical vagus nerve and optimization results. Left: Cross section of the modeled nerve and six electrode contacts. Blue fascicles are the targets. Red dots indicate active axons as evaluated in NEURON. Right: Stimulus waveforms for each of the six electrode contacts as determined by the DE optimization. 100% of target fascicles were activated with 0% off-target fascicles activated.

FIG. 15 illustrates a human cervical vagus nerve and optimization results. Left: Cross section of the modeled nerve and six electrode contacts. Blue fascicles are the targets. Red dots indicate active axons as evaluated in NEURON. Right: Stimulus waveforms for each of the six electrode contacts as determined by the DE optimization. 88.9% of target fascicles were activated with 0% off-target fascicles activated.

FIG. 16 illustrates an example scheme of a gradient-based optimization for parameter selection. Convergence for a gradient-based optimization approach may for example be defined in terms of the evaluated loss function not decreasing by a pre-specified amount for a pre-specified number of optimization step iterations.

FIG. 17 illustrates results of a gradient descent optimization in a selectivity task where the stimulation waveforms have not been specified. The output parameters (i.e., the model parameters being optimized) were the current amplitudes in mA at every timestep for each of the 6 electrode contacts for a 2.5 ms simulation with a timestep of 0.005 ms for a total of 3000 optimizable parameters. The right-hand panel shows the cross section of a pig vagus nerve, where the inner shapes denote fascicles (bundles of axons), electrode contacts around the nerve are numbered 1-6; target fascicles are indicated in blue, with a single axon (5.7 µm MRG) placed at the centroid of each fascicle. Optimized waveforms for each of the contacts are on the left, with numbers corresponding to the electrode contact from which the waveform is delivered. The neuronal response as evaluated using the biophysical model (NEURON) is on the right - red dots indicate activated axons (i.e., axons that generated a propagating action potential), and green indicates axons that were not activated. In this instance, the method achieved perfect selectivity (i.e., only axons in the target fascicles generated a propagating action potential).

FIG. 18 illustrates implementations applying optimization results using a binary classification surrogate of FIG. 2 to evaluate the loss function. The tissue morphology is a cylindrical 1 mm radius nerve consisting of a linear, isotropic, semi-infinite, homogeneous, frequency-independent medium with a resistivity of 1000 Ω-cm. Illustrated is a cross section through the nerve, blue and red dots are locations of axons (300 total) running parallel to the nerve and the 8 yellow dots are point-source electrode contacts at the nerve boundary. The target axon population is indicated with a dashed black line. Some embodiments are optimized for monophasic pulses with fixed pulse widths set at 0.3 ms, with amplitudes bounded between 2 and 0 mA and delay bounded between 0.5 and 0.8 ms. Blue dots signify axons that have not produced an action potential and red dots indicate axons that have produced an action potential. The final 8 stimulus waveforms (one per contact) under three different implementations A, B, and C are plotted around the nerve circumference. A. Using only what the surrogate believes is the best solution in the final population yields a true (NEURON-validated) final loss of 0.67, corresponding to 47.1% target population activated and 2.5% off-target population activated in 3 min 30 s. B. Evaluating the top 100 final population members (as judged by the surrogate) in NEURON yields a best solution with loss of 0.55, corresponding to 58.8% target population activated and 1.8% off-target population activated in 8 min 24 s (parallelized across 500 CPU cores). C. Using the top 100 final population members (as judged by the surrogate) to initialize the differential evolution algorithm and run for another 25 generations using NEURON to evaluate the loss function yields a best solution with final loss of 0.36, corresponding to 82.4% target population activated and 1.8% off-target population activated in 1 hr 59 min (parallelized across 500 CPU-cores). This performance is better than randomly initializing the population with Latin hypercube sampling and running the differential evolution optimizer with the biophysical model, which yields a solution with a final loss of 0.565 in 1 hr 56 min.

FIG. 19 illustrates an example of a recommender system to generate stimulation parameters. The recommender system is implemented as a fully-connected neural network (recommender network). Also illustrated is the associated procedure to train the recommender system by gradient descent using the derivatives of the recommender system weights with respect to a differentiable loss function (∇_{w}L). The loss function (L) is calculated with respect to the output (Prediction) of a differentiable model of the neuronal response (surrogate model).

FIG. 20 illustrates graphs of threshold errors in a model of pig VNS, between the trained surrogate model and the full NEURON simulations for sawtooth stimulus waveforms. Each plot corresponds to an individual source-fascicle pair (i.e., all fibers used to generate the data for a given plot were placed in the same fascicle within the nerve and received stimulation from the same electrode contact). Horizontal dashed lines correspond to +5%, 0% and -5% threshold error.

FIG. 21 illustrates graphs of threshold errors in a model of pig VNS, between the trained surrogate model and the full NEURON simulations for exponential stimulus waveforms. Each plot corresponds to an individual source-fascicle pair (i.e., all fibers used to generate the data for a given plot were placed in the same fascicle within the nerve and received stimulation from the same electrode contact). Horizontal dashed lines correspond to +5%, 0% and -5% threshold error.

FIG. 22 illustrates graphs of threshold errors in a model of pig VNS, between the trained surrogate model and the full NEURON simulations for Gaussian stimulus waveforms. Each plot corresponds to an individual source-fascicle pair (i.e., all fibers used to generate the data for a given plot were placed in the same fascicle within the nerve and received stimulation from the same electrode contact). Horizontal dashed lines correspond to +5%, 0% and -5% threshold error.

FIG. 23 illustrates graphs of threshold errors in a model of pig VNS, between the trained surrogate model and the full NEURON simulations for sinusoidal stimulus waveforms. Each plot corresponds to an individual source-fascicle pair (i.e., all fibers used to generate the data for a given plot were placed in the same fascicle within the nerve and received stimulation from the same electrode contact). Horizontal dashed lines correspond to +5%, 0% and -5% threshold error.

### DETAILED DESCRIPTION

Section headings as used in this section and the entire disclosure herein are merely for organizational purposes and are not intended to be limiting.

All publications, patent applications, patents and other references mentioned herein are incorporated by reference in their entirety.

### 1. Definitions

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. By way of example, “an element” means at least one element and can include more than one element. The present disclosure also contemplates other embodiments “comprising,” “consisting of” and “consisting essentially of,” the embodiments or elements presented herein, whether explicitly set forth or not. As used herein, “and/or” refers to and encompasses any and all possible combinations of one or more of the associated listed items, as well as the lack of combinations where interpreted in the alternative (“or”).

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated. Recitation of ranges of values herein are merely intended to serve as a shorthand method of referring individually to each separate value falling within the range, unless otherwise-Indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. “About” or “approximately” is used to provide flexibility to a numerical range endpoint by providing that a given value may be “slightly above” or “slightly below” the endpoint without affecting the desired result.

“Subject” and “patient” as used herein interchangeably refers to any vertebrate, including, but not limited to, a mammal (e.g., cow, pig, camel, llama, horse, goat, rabbit, sheep, hamsters, guinea pig, cat, dog, rat, and mouse, a non-human primate (e.g., a monkey, such as a cynomolgus or rhesus monkey, chimpanzee, etc.) and a human). In some embodiments, the subject may be a human or a non-human. In one embodiment, the subject is a human. The subject or patient may be undergoing various forms of treatment.

“Treat,” “treating” or “treatment” are each used interchangeably herein to describe reversing, alleviating, or inhibiting the progress of a disease and/or injury, or one or more symptoms of such disease, to which such term applies. Depending on the condition of the subject, the term also refers to preventing a disease, and includes preventing the onset of a disease, or preventing the symptoms associated with a disease. A treatment may be either performed in an acute or chronic way. The term also refers to reducing the severity of a disease or symptoms associated with such disease prior to affliction with the disease. Such prevention or reduction of the severity of a disease prior to affliction refers to administration of a treatment to a subject that is not at the time of administration afflicted with the disease. “Preventing” also refers to preventing the recurrence of a disease or of one or more symptoms associated with such disease.

“Therapy” and/or “therapy regimen” generally refer to the clinical intervention made in response to a disease, disorder or physiological condition manifested by a patient or to which a patient may be susceptible. The aim of treatment includes the alleviation or prevention of symptoms, slowing or stopping the progression or worsening of a disease, disorder, or condition and/or the remission of the disease, disorder or condition.

The term “effective amount” or “therapeutically effective amount” refers to an amount sufficient to effect beneficial or desirable biological and/or clinical results.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, neurobiology, microbiology, genetics, electrical stimulation, neural stimulation, neural modulation, and neural prosthesis described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

### 2. Overview

One aspect of the present disclosure provides novel systems and methods to estimate the response of neurons to electrical stimulation and to determine the optimal electrode geometry and parameters of stimulation to activate or block specific targeted groups of neurons without activation or block of non-targeted groups of neurons. These methods provide essential tools for the design of experimental and therapeutic interventions. The disclosed system enables a user to specify the neurons targeted for activation or block (e.g., types, locations), and the method identifies the electrode geometry and/or stimulation parameters to achieve neuron-selective activation or block.

The development of bioelectronic therapies is limited by inadequate activation of targeted neurons and spillover of activation to non-targeted neurons. More fundamentally, the relationship between applied stimuli and the complement of neurons that are activated or blocked, how this relationship varies across individuals and between species, and how these relationships can be controlled remain largely unknown.

Development of effective, efficient, and selective neural activation and block requires exploration and understanding of a large multi-dimensional parameter space, including stimulation waveform, amplitude, frequency, and electrode design. Further, the optimal or even appropriate solutions are likely to vary across nerves, species, and individuals. Computational models, especially those that represent the anatomical, morphological, and biophysical properties of the nerves of interest, enable efficient exploration of this parameter space and rigorous optimization of application-specific parameters. Model-based approaches are effective, for example, for analysis and design of electrodes for selective stimulation of peripheral somatic nerves. Further, such models enable investigation of the mechanisms underlying complex non-linear phenomena, such as conduction block.

Disclosed herein are novel methods for estimating electrical activation and block of neurons (hereafter referred to as ‘surrogate’ models, or surrogate network). The method enables determination of the distribution of activated and blocked neurons, including neurons with a specific subthreshold response, with orders of magnitude less computational time compared to standard biophysical models. The method serves as an essential engineering design tool where iterative parametric in vivo studies are largely impractical.

In contrast to conventional threshold-based estimators of the neural response to applied signals, where the estimate consists of whether a neuron has crossed a pre-computed ‘firing threshold’ for a specific stimulus waveform, the method retains generality in its estimates and predicts a broad range of complex non-linear phenomena exhibited by neurons in response to arbitrary waveforms in arbitrary electric fields. Examples include but are not limited to: interactions between propagating action potentials (where action potentials will meet and mutually annihilate) and onset response and conduction block resulting from a high (kHz)-frequency signal (where a neuron will initially fire action potentials and then go silent and prevent propagation of action potential across the region beneath the stimulating electrode when extracellularly stimulated with a kHz frequency signal).

Models of neural stimulation are traditionally used in the forward direction, where the user specifies the inputs (e.g., electrode geometry, stimulation waveform, stimulation intensity), and the model calculates the response of neurons (e.g., activation, block). As detailed herein, an inverted model is where the user specifies the neurons targeted for activation or block, and the model calculates the appropriate electrode geometry and/or stimulation parameters. However, because the response of neurons to stimulation is highly non-linear, e.g., doubling the intensity of stimulation does not lead to a doubling of neuron activation or block, this cannot be accomplished by simply inverting the forward model. Rather, disclosed herein are gradient-free global optimization and gradient-based optimization methods to determine input parameters that produce target outcomes in the models of stimulation. In combination with the novel surrogate methods to estimate neural responses, these approaches enable solving higher-dimensional problems than previously achieved, and for analogous problems, they are solved with reduced computational cost and time. Additionally, disclosed herein is the application of machine learning methods alongside the surrogate models disclosed herein to learn neural networks (hereafter referred to as ‘recommender’ networks) to recommend directly model-specific values of programmable parameters to achieve selective activation and block under a variety of constraints.

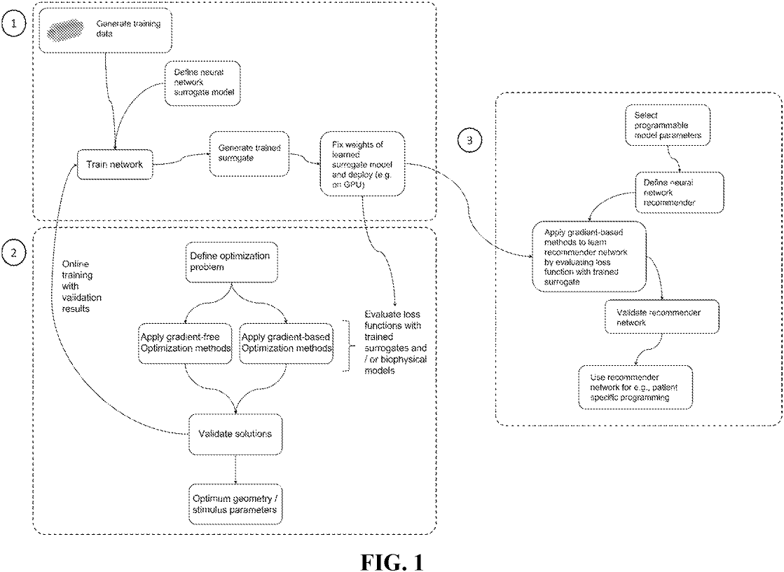

With reference to FIG. 1, the overall system for model-based selection of electrode geometry and/or stimulus parameters is illustrated. Broadly, the system has two components: (1) design, implementation, and execution of surrogate computational models of a neuron’s biophysical response to extracellular stimulation, and (2) design, implementation, and execution of processes for solving an optimization problem related to selective nerve stimulation using the learned surrogates in addition to / instead of standard biophysical cable models. Additionally, the system includes a method (3) to use the surrogate computational models to train recommender networks to generate model-specific stimulation parameter values to achieve selective activation / block of user-defined targets.

The inventions described herein are used to support real-time prediction of neural responses to stimulation in clinical settings. They are used to implement systems and software for designing and programming neuromodulation devices, with or without patient-specific models. They are used to implement modeling platforms for research and development to analyze existing therapies and design new therapies, including the geometry of the neurostimulators themselves, the positioning of the neurostimulator with respect to the target excitable tissue, and the waveforms and patterns of stimulation used when delivering stimulation. The recovery of complex non-linear phenomena by the surrogate models means that the surrogate models can be used to investigate interactions between non-local effects to achieve therapeutic outcomes (e.g., downstream filtering of action potentials by high frequency stimulation).

### 3. Neuron Surrogate Models

A surrogate model for stimulation of a neuron takes as input a sequence of numerical vectors representing the extracellular electric field as it varies over the course of stimulation and returns a sequence of numerical vectors representing the evolving state of the neuron over the same period (e.g., transmembrane voltage(s) or current(s), or variables representing the state of conduction of individual ion channels, or a metric relating the probability of some dynamic event (e.g., an action potential) occurring within the modeled time window, or any combination and / or function thereof). In the surrogate model, this mapping may be performed and/or augmented by a neural network or other machine learning or artificial intelligence construct (e.g., kernel methods such as support vector machines, Kalman filters, etc.).

The process for learning a surrogate neuron model consists of training a neural network to reproduce the responses of biophysical neuron models to extracellular electrical stimulation. In some embodiments, biophysical models are implemented and solved using the NEURON simulation environment, although the methods apply to other frameworks, software packages, or programming languages for simulating biophysical models, e.g., Arbor, Brian 2, or MATLAB. In some embodiments, other simplified models of neurons are implemented and solved.

More specifically, in some embodiments, the sequence of numerical vectors supplied as inputs representing the extracellular fields are the extracellular potentials sampled along the compartments of each modeled neuron. In some embodiments, these potentials are extracted from a finite element model of anatomically realistic nerve morphologies instrumented with an electrode or may be computed using simpler models such as point and line source idealizations, or other estimates of the electric field. The state(s) returned by the surrogate model can represent spatiotemporal dynamics (e.g., gating variable values across time and space) and more abstract features (e.g., to predict the probability of generating an action potential).

For some embodiments described herein, the surrogate model is of the 5.7 µm diameter MRG axon model, a well-established biophysical multicompartment myelinated axon model. In these implementations, the input extracellular electric field is sampled along contiguous sequences of nodes of Ranvier, and the outputs consist of either the probability of an action potential occurring within the modeled time window or ion channel gating variable values at the same nodes at which the field was sampled for every modeled timestep. The methods outlined apply more generally to data sampled at any set of discrete compartments in any multicompartment neuron model, myelinated or unmyelinated, of any diameter, of arbitrary length and diameter, and may be used to predict any set of dynamic variables (including the transmembrane potential, transmembrane current, gating variables) for any set of compartments (not necessarily the same compartments at which the extracellular field was sampled), as well as at different timestep resolutions than was used for solving the biophysical model.

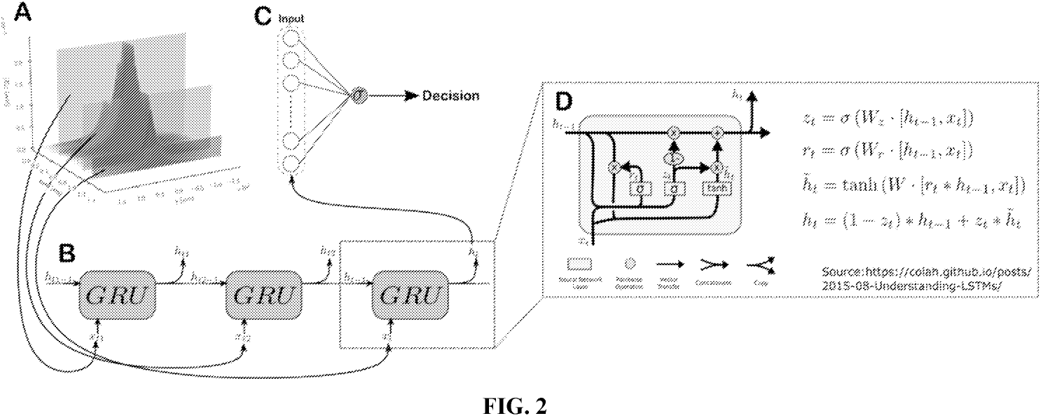

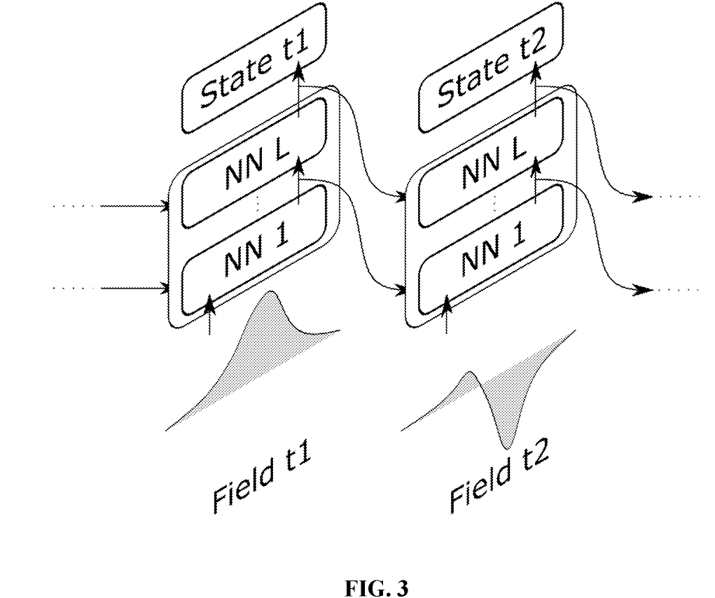

Classification Surrogate: With reference to FIG. 2, a neural network is designed, implemented, trained, and tested to predict whether an action potential has occurred in a 5.7 µm MRG axon (hereafter referred to as ‘classification surrogate’). A single fully connected Gated Recurrent Unit (GRU) is used to extract features from the input sequence of extracellular voltages that were passed to a fully connected sequence of layers for classification, whereby the output is a value between 0 (did not produce an action potential) and 1 (did produce an action potential) describing the probability that an action potential was generated.

Gating Variable Surrogate: With reference to FIG. 3, a neural network is designed, implemented, trained, and tested to predict the gating variable responses as a function of time at each node of Ranvier for a 5.7 µm MRG axon (hereafter referred to as ‘gating variable surrogate’). A variation of a stacked Long Short-Term Memory (LSTM) network is used in some embodiments, where L ≥ 1 in FIG. 3 is the number of independent LSTM networks in the stack (labeled NN in FIG. 3). In both cases, other recurrent neural network architectures may be substituted for those illustrated, such as an Elman network, another gated recurrent neural network such as GRU, fully convolutional architectures for sequence analysis and/or synthesis such as WaveNet, or attention-based models such as a Transformer. In some embodiments, the networks are implemented in PyTorch v1.9, though other deep-learning frameworks such as TensorFlow may also be used.

For the gating variable surrogate, let N be the number of nodal compartments being modeled, and t the timestep. Then xₜ ∈ ℝ^{N} is the distribution of extracellular potentials sampled at those N nodes at time t. A single neural network (NN) within the stack illustrated in FIG. 3 applies updates recursively on a pair of latent vectors h ∈ ℝ^{F×N} and c ∈ ℝ^{F×N} according to the following rule (a variation on the LSTM network model), given as EQNS.1-8.

$${i}_{t} = σ \left({W}_{i} ∗ \left[{h}_{t - 1} , {x}_{t}\right] + {b}_{i}\right) \qquad \text{[EQN. 1]}$$

$${f}_{t} = σ \left({W}_{f} ∗ \left[{h}_{t - 1} , {x}_{t}\right] + {b}_{f}\right) \qquad \text{[EQN. 2]}$$

$${o}_{t} = σ \left({W}_{o} ∗ \left[{h}_{t - 1} , {x}_{t}\right] + {b}_{o}\right) \qquad \text{[EQN. 3]}$$

$${\tilde{c}}_{t} = t a n h \left({W}_{c} ∗ \left[{h}_{t - 1} , {x}_{t}\right] + {b}_{c}\right) \qquad \text{[EQN. 4]}$$

$${\hat{c}}_{t} = {f}_{t} ⊙ {c}_{t - 1} + {i}_{t} ⊙ {\tilde{c}}_{t} \qquad \text{[EQN. 5]}$$

$${\hat{h}}_{t} = {o}_{t} ⊙ tanh \left({c}_{t}\right) \qquad \text{[EQN. 6]}$$

$${c}_{t} = r \times {c}_{t - 1} + \left(1 - r\right) \times {\hat{c}}_{t} \qquad \text{[EQN. 7]}$$

$${h}_{t} = r \times {h}_{t - 1} + \left(1 - r\right) \times {\hat{h}}_{t} \qquad \text{[EQN. 8]}$$

where

$$σ \left(p\right) = \frac{1}{1 + {e}^{- p}}$$

and

$$tanh \left(p\right) = \frac{{e}^{p} - {e}^{- p}}{{e}^{p} + {e}^{- p}} .$$

[p, q] indicates the concatenation of tensors p and q along the feature axis; the input xₜ, representing the forcing term (i.e., the imposed extracellular potentials) at time t, has only one feature (the potential), while the hidden and cell states hₜ and cₜ may have arbitrarily many features F (i.e., the number of features is a model hyperparameter chosen to tune the capacity of the model). The vector r ∈ ℝ^{F} is learned during training and is a parameter that enables the network to globally adjust the rate at which each feature of the internal states changes every timestep. The hyperparameter L defines how many such recursive mappings between vector spaces can be applied sequentially to learn more sophisticated relationships between input and output. Only the hidden state hₜ is propagated vertically between LSTMs (NNs) in the stack (whereas both hₜ and cₜ are propagated forward in time). Empirically, a 3 layer LSTM network (i.e., L = 3 in FIG. 3) performs well for reproducing node-level dynamics of the MRG axon; in some implementations, these dynamics are gating variables representing the state of conduction of individual membrane ion channels, though the dynamics could relate to transmembrane voltage(s) or current(s) or any combination(s) / function(s) thereof. However, different capacities may be appropriate for different models of a neuron. In the final layer of the implementation, the tanh nonlinearity for the hidden state hₜ is replaced with σ to reflect the natural bounds for ion channel gating variables (between 0 and 1).

W ∗ p indicates discrete 1D convolution between weights W and tensor p. W and b in the equations above are parameters that are learned during training. In contrast to a standard LSTM network, in which state updates are mediated by fully connected networks, an LSTM architecture consisting of 1D convolutional primitives is used. This confers various structural properties to the network that are important to preserve from the biophysical multicompartment neuron model. Specifically, one such structural property is that the network can handle variable input sizes, i.e., arbitrary numbers of compartments of the neuron can be modeled, and once the model is trained, it can predict responses from different numbers of compartments. Furthermore, using convolutions means that the models disclosed herein have translational symmetry (e.g., can transpose the field along the length of modeled neuron and reproduce the same dynamical behavior at those different locations). Additionally, unlike in the fully connected network case, where each modeled compartment contributes to the immediate future state of every other, convolutions integrate information locally: the width of the 1D convolutional kernel determines from how many adjacent compartments the future state will be calculated. This is key for achieving node-level resolution of dynamics (i.e., transmembrane voltage(s) or current(s), or variables representing the state of conduction of individual ion channels) that generalizes well. In some implementations, a kernel of size 3 is used, integrating information across three adjacent nodes, where the future state(s) of node i will depend on the current state(s) of nodes i - 1, i, and i + 1. Finally, in some implementations, symmetry is enforced in the convolutional kernels along their width (i.e., the node / compartment axis); this is critical for avoiding the accumulation of noise in the system, recovering the fast rates of change in the system states related to phenomena such as action potentials, and improving stability.

The neural networks are trained on data generated by solving biophysical neuron models using standard numerical integration methods or by simulating other types of models of neuronal responses. The extracellular electric field generated by stimulation that is applied to the biophysical neuron models may be solved in biophysically realistic field models, for example by solving a finite element model of a tissue morphology derived from histology. The electric fields may also be solved in idealized field models, using for example point- or line-source field models. Alternatively, the electric fields may be generated entirely from a synthetic probability distribution specifying properties such as the peak amplitude, steepness, and symmetry. The generated dataset consists of field-response pairs, where the field is a sequence of vectors representing the electric field at each compartment of the model neuron, and the response may be, e.g., a sequence of vectors representing the states of the neuron to be predicted, for example the transmembrane voltage(s) or current(s), or gating variables representing the state of conduction of individual ion channels, or a value 0 or 1 corresponding to the absence or presence of an action potential, or combinations and/or functions thereof.

In some gating variable surrogate implementation, training data samples (i.e., individual field-response pairs) are generated for axon fibers positioned at the centroids of fascicles within a morphologically realistic pig vagus nerve model consisting of 31 fascicles, while in the classification surrogate implementation, training data samples are generated for axon fibers in a homogeneous isotropic nerve model. Data collection consists of randomly generating parameters for monophasic rectangular pulses for each of the electrode contacts arranged circumferentially around the nerve. In some embodiments, Latin hypercube sampling is used with sample size of 32 over the intervals [-20, 20] mA for amplitude, [0, 2] ms for pulse width, and [0, 2] ms for delay. However, differently parameterized waveforms (e.g., biphasic pulses, sums of sinusoids, spline-interpolated functions), other parameterization values, and any combination thereof, as well as different sampling methods (e.g., normal random sampling, uniform random sampling, Sobol sampling), may be used. The data collected could be for one or multiple sizes of neurons. The axonal responses to each sampled parameter set are simulated over 10 ms (though other time windows may be simulated) and the presence of an action potential is determined by checking whether the transmembrane potential crossed 0 mV at a nodal compartment 1 mm from the end of the simulated fiber. For generating training data for the gating variable surrogate, the m, p, h, and s gating variables are recorded at 87 nodal compartments for each axon. This is repeated 128 times for a total of 126,976 axon simulations constituting 126,976 training samples.

The networks are trained by gradient descent using batches of data (i.e., a subset of field-response pairs from the entire dataset) randomly sampled from this dataset. The loss function for the gating variable surrogate is defined as the root mean squared error between the network prediction and the true gating variable values, although other loss functions such as the mean absolute error or mean squared error may be used. The loss function for the classification surrogate is the binary cross entropy loss, although other functions such as the hinge loss or squared hinge loss may be used. A variety of algorithms exist for learning neural networks by gradient descent; in the implementation, the RMSprop optimizer with a learning rate of 1e-4 on batches of size 192 produced good results. However, other optimization algorithms such as (but not limited to) Adam (Kingma and Ba, 2014), Adagrad (Duchi et al., 2011) and RAdam (Liu et al., 2021) may be used; the appropriate learning rate will depend on the choice of optimizer, and the appropriate batch size will depend on the specific computing hardware being used to train the model, the specific learning rate of the optimization algorithm, and the specific dataset used for training. The surrogates may be learned and deployed on massively multithreaded hardware, e.g., the implementations run on Nvidia RTX A5000 GPUs, although the networks may be deployed on, e.g., other CUDA GPUs with minimal additional implementation cost. When deployed on such hardware, the surrogate models generate predictions of axonal responses in novel field models at up to 500x increased speed for the gating variable surrogate implementation and up to 2000x increased speed for the classification surrogate implementation compared to a single-threaded biophysical axon implementation.

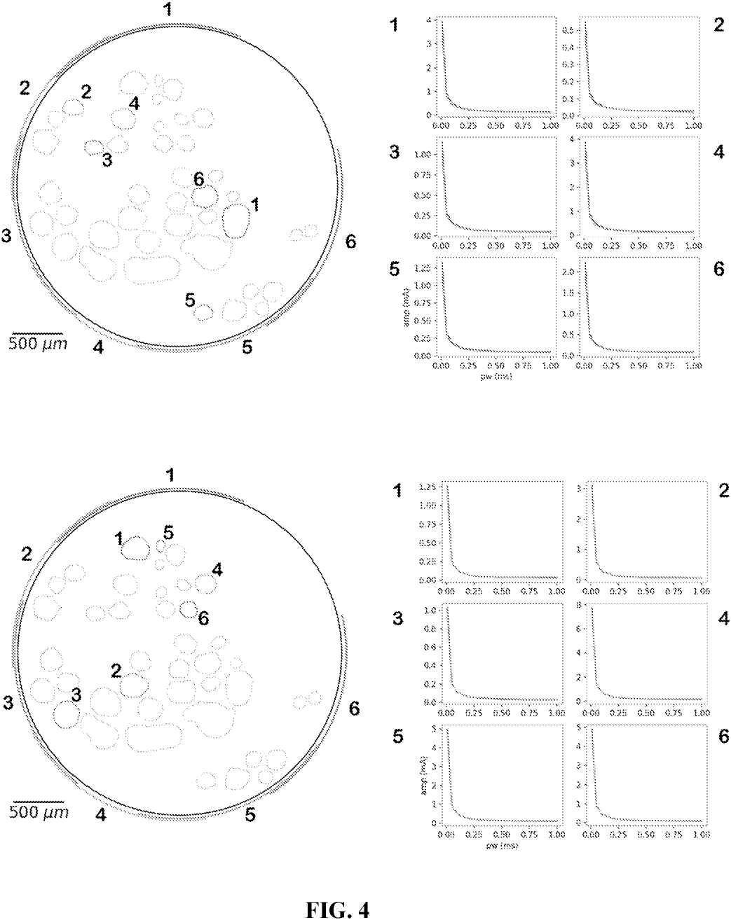

FIG. 4 illustrates the gating variable surrogate model’s predictions of strength-duration relationships, i.e., the threshold stimulus current as a function of the stimulus pulse duration, in a new field model (i.e., distinct from the one used to generate training data) for monophasic and biphasic stimulation pulses with comparison to solving the non-linear differential equations describing the biophysical model. ‘Strength-duration relationship’ refers to the minimum stimulation amplitude (in mA of current), i.e., strength, required to generate a propagating action potential in a neuron for a specific duration stimulus, for a given source-neuron pair.

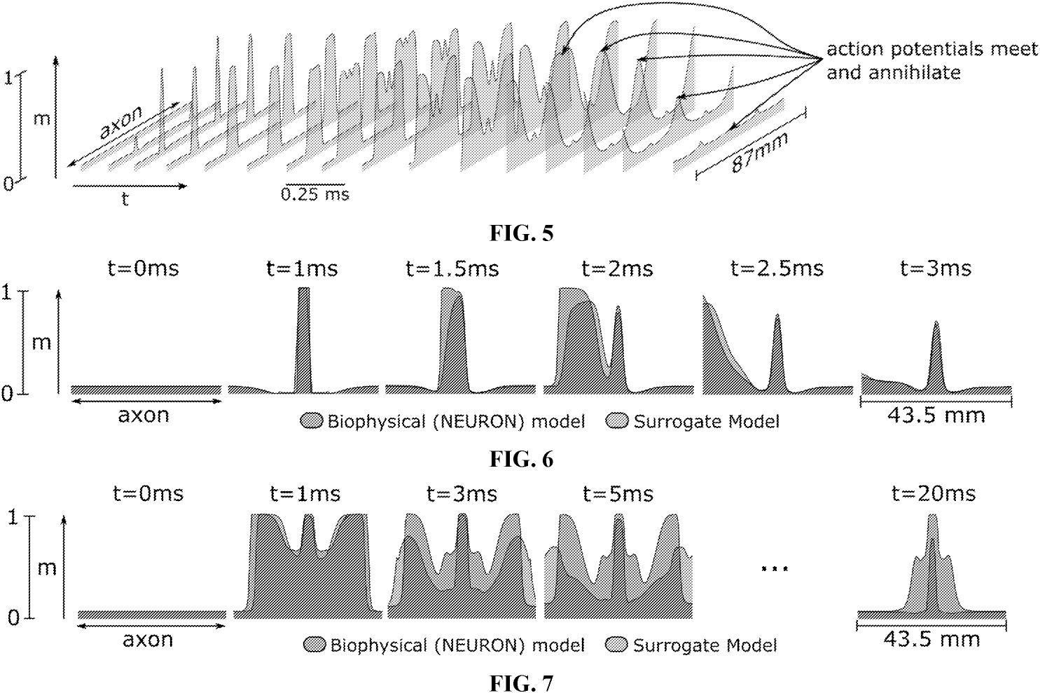

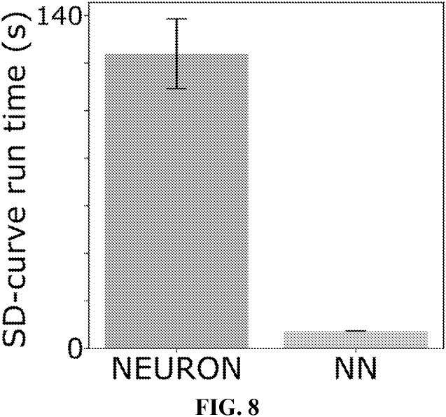

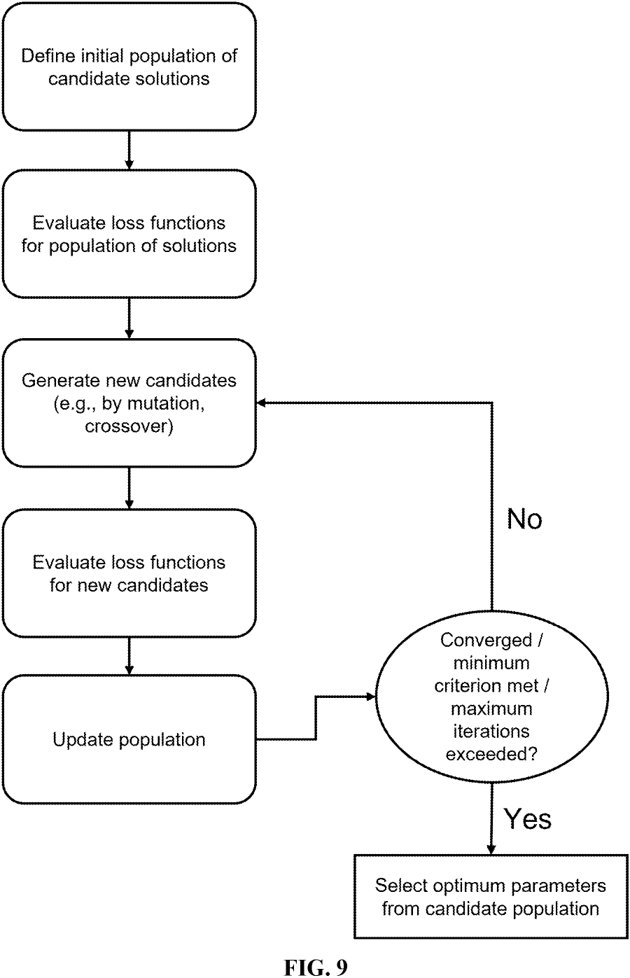

FIGS. 5-7 illustrate complex non-linear phenomena recovered by the gating variable surrogate model, specifically a 3-layer recurrent neural network, as illustrated in FIG. 3, trained to reproduce the m, h, p, and s gating variables at the nodes of Ranvier of the MRG axon model across space and time. FIG. 8 compares performance in terms of wall-clock calculation time for constructing strength-duration curves using the biophysical model vs. the surrogate model.

Cable Surrogate: In one surrogate model implementation, a simplified cable model is designed, implemented, trained, and tested to predict the full system state (gating variables and transmembrane potential) as a function of time at each node of Ranvier for 5.7 to 14 µm diameter MRG axons (hereafter referred to as ‘cable surrogate’).

In some embodiments, only the nodal compartments of the complete MRG fiber model are retained and the myelin is assumed to be perfectly insulating. Other geometric abstractions (e.g., explicitly modeling the internode, adding additional layers of extracellular field, and any combinations and variations thereof) may be adopted. In some embodiments, a forward Euler discretization is applied with staggered timestep to solve the resulting system of differential equations. Shown here for node n, advancing from time t to t+dt, first update gating parameter values as EQN. 9.

$$\begin{matrix}m \left(n , t + 0.5 d t\right) = m \left(n , t - 0.5 d t\right) + d t \times \\ \left({α}_{m} \left(V m \left(n , t\right)\right) \left(1 - m \left(n , t - 0.5 d t\right)\right)\right) - \\ \left({β}_{m} \left(V m \left(n , t\right)\right) m \left(n , t - 0.5 d t\right)\right)\end{matrix} \qquad \text{[EQN. 9]}$$

Likewise for h, p, and s, where αₓ and βₓ are independent rate constant functions for gating variable ‘x’. Using m to indicate m(n, t + 0.5dt) (likewise for all other gating variables h, p, and s) and Vm to indicate Vm(n, t), gives EQNS. 10 and 11.

$$\begin{matrix}{I}_{i o n} = {\overline{g}}_{N a f} {m}^{3} h \left(V m - {E}_{N a}\right) + {\overline{g}}_{N a p} {p}^{3} \left(V m - {E}_{N a}\right) + \\ {\overline{g}}_{K} s \left(V m - {E}_{N a}\right) + {g}_{L} \left(V m - {E}_{L}\right)\end{matrix} \qquad \text{[EQN. 10]}$$

$$\begin{matrix}V m \left(n , t + d t\right) = V m \left(n , t\right) + d t \times \frac{1}{C m} \times \\ \left(\frac{1}{R a} \times {D}^{2} \left(\left[V m \left(n , t\right) , V e \left(n , t\right)\right]\right) - {I}_{i o n}\right)\end{matrix} \qquad \text{[EQN. 11]}$$

Where D²([Vm(n,t),Ve(n,t)]) is the sum of second spatial differences of Vm and Ve centered on node n, implemented here as a linear convolution over concatenated Vm and Ve vectors with kernel .

Other explicit (e.g., higher order Runge-Kutta) and / or implicit (e.g., implicit Euler) numerical integration methods may be used, and any combinations or variations thereof. Other neuronal models with different biophysics or different simplified representations may likewise be adapted, with appropriate adjustment and / or addition / subtraction of the relevant set of ionic conductances and associated rate constant functions.

In some embodiments, backpropagation and gradient descent are used to apply the appropriate parametric adjustments to achieve better agreement between the simplified cable model and the complete (NEURON implemented) biophysical model. In some embodiments, the models are implemented in PyTorch v1.9 though other deep learning frameworks, such as TensorFlow, and automatic differentiation packages more generally, may be used. In some embodiments, the peak ionic conductance values (g̅_{Naf}, etc.), axial resistivity, membrane capacitance, and the values of the convolutional kernel used to compute D² were assigned as optimizable parameters. The method freely permits other parameters (such as the coefficients of the rate constant equations, equilibrium potentials, etc.) and all subsets and combinations thereof to also be selected for optimization. Furthermore, any functions constituting the set of equations describing the cable model (for example, the rate constant functions) may in part or in whole be substituted with generic function approximators implemented as a neural network or other machine learning construct and trained alongside the other model components. Likewise, the biophysical parameters themselves (for example, though not limited to, the axial resistivity) may be implemented as functions of inputs to the model (for example, though not limited to, the fiber diameter), either explicitly parameterized or approximated with a neural network or other machine learning construct, with the resulting functions also optionally trained alongside the rest of the model.

In some embodiments, a scaling correction is implemented for Ve as a 3^{rd} order polynomial function of the fiber diameter which was fit using the least squares method after the model had been trained. The scaling correction may be implemented as any parametric function or neural network or other machine learning construct and may also be trained alongside the rest of the model parameters or omitted altogether.

As with the other surrogate models described herein, the cable surrogate is trained on data generated by solving biophysical neuron models using standard numerical integration methods. The extracellular electric field generated by stimulation that is applied to the biophysical neuron models may be solved in biophysically realistic field models, for example by solving a finite element model of a tissue morphology derived from histology. The electric fields may also be solved in idealized field models, using for example point- or line-source field models. Alternatively, the electric fields may be generated entirely from a synthetic probability distribution specifying properties such as the peak amplitude, steepness, and symmetry. The generated dataset consists of field-response pairs, where the field is a sequence of vectors representing the electric field at each compartment of the model neuron, and the response is a sequence of vectors representing the states of the neuron to be predicted, for example the transmembrane voltages and gating parameter values, or combinations and / or functions thereof.

In some embodiments of the cable surrogate, training data samples (i.e., individual field-response pairs) are generated for axon fibers with diameters randomly sampled from a uniform distribution over [5.7 µm, 14 µm]. Axons are positioned at the centroids of fascicles within a morphologically realistic pig vagus nerve model consisting of 53 fascicles. Data collection consisted of randomly generating parameters for monophasic rectangular pulses for each of the electrode contacts arranged circumferentially around the nerve. In some embodiments, Latin hypercube sampling is used with sample size of 32 over the intervals [-0.1, 0.1] mA for amplitude, [0, 2] ms for pulse width, and [0, 2] ms for delay. However, differently parameterized waveforms (e.g., biphasic pulses, sums of sinusoids, spline-interpolated functions) and any combination thereof, as well as different sampling methods (e.g., normal random sampling, uniform random sampling, Sobol sampling), may be used. The axonal responses to each sampled parameter set were simulated over 7.5 ms (though other time windows may be simulated). For generating training data for the cable surrogate, the m, p, h, and s gating variables as well as the membrane potential were recorded at 53 nodal compartments for each axon. This was repeated 32 times for a total of 54,272 axon simulations constituting 54,272 training samples.

The model is trained by gradient descent using batches of data (i.e., a subset of field-response pairs from the entire dataset) randomly sampled from this dataset. The loss function for the gating cable surrogate was defined as the mean squared error between the cable surrogate prediction and the true transmembrane voltage and gating variable values, although other loss functions such as the mean absolute error or root mean squared error may be used.

A variety of algorithms exist for learning differentiable functions by gradient descent. In some implementations, a truncated backpropagation through 50 timesteps and gradient descent with the RMSprop update rule with a learning rate of 1e-6 on batches of size 53 produced good results. However, other optimization algorithms such as (but not limited to) Adam (Kingma and Ba, 2014), Adagrad (Duchi et al., 2011) and Radam (Liu et al., 2021) may be used the appropriate learning rate will depend on the choice of optimizer, and the appropriate batch size will depend on the specific computing hardware being used to train the model, the specific learning rate of the optimization algorithm, and the specific dataset used for training. The surrogates may be learned and deployed on massively multithreaded hardware, e.g., the implementations run on Nvidia RTX A5000 GPUs, although the networks may be deployed on, e.g., other CUDA GPUs with minimal additional implementation cost. When deployed on such hardware, the cable surrogate generated predictions of axonal responses in novel field models at up to 10,000x increased throughput compared to a single-threaded biophysical axon implementation.

The cable surrogate is fully differentiable with respect to all parameters, and therefore fully compatible with all optimization methods described previously (gradient based and gradient free).

Embodiments of the present disclosure include a method for estimation of a neuronal response, the method comprising: inputting a sequence of numerical input vectors corresponding to an electric field over a period of neuronal stimulation into a surrogate model of neuronal stimulation. The surrogate model of neuronal stimulation outputs a sequence of numerical output vectors corresponding to one or more changes in neuronal response during the stimulation period.

As described further herein, in some embodiments, the sequence of numerical input vectors comprise a spatiotemporal distribution of extracellular potentials measured, calculated, or estimated for the period of neuronal stimulation.

As described further herein, in some embodiments, the one or more changes in neuronal response corresponding to the sequence of numerical output vectors comprise at least one of: transmembrane voltage, total transmembrane current, transmembrane current for one or more ions, conduction state of an ion channel, detection of one or more action potentials, a probability of a dynamic event, gating variable values, or any combination thereof.

As described further herein, in some embodiments, the surrogate model comprises a machine learning component and/or an artificial intelligence component comprising at least one of: recurrent neural network, convolutional neural network, fully connected neural network, decision tree, random forest, support vector machine, linear regression, logistic regression, nearest neighbors, or any combination thereof.

As described further herein, in some embodiments, the method further includes training the surrogate model of neuronal stimulation with data from simulation of a biophysical model or a simplified model.

As described further herein, in some embodiments, the sequence of numerical input vectors is from a model of an anatomically realistic nerve morphology with an electrode.

As described further herein, in some embodiments, the surrogate model is a classification surrogate that determines a probability that an action potential has occurred.

As described further herein, in some embodiments, the surrogate model determines a strength-duration relationship, determines a relationship between activation thresholds and neuron size, determines a relationship between block thresholds and neuron size, determines subthreshold response, identifies unidirectional propagation, identifies bidirectional propagation, determines propagation speed, determines the number of evoked action potentials, determines the time of action potential initiation, determines the location of action potential initiation, determines a gating variable, captures interaction between propagating action potentials, captures interaction between a subthreshold change in membrane state and a propagating action potential, captures interaction between changes in membrane state and responses to stimulation, predicts a state of conduction block, or any combination thereof.

As described further herein, in some embodiments, the surrogate model is a cable surrogate that includes nodal compartments of a complete neuron model.

As described further herein, in some embodiments, the surrogate model is fully differentiable with respect to all parameters.

As described further herein, in some embodiments, the sequence of numerical input vectors corresponds to an arbitrary spatial distribution of extracellular potentials.

As described further herein, in some embodiments, the sequence of numerical input vectors correspond to an arbitrary temporal shape extracellular potentials.

As described further herein, in some embodiments, the design of the surrogate model predicts the response of one or multiple sizes of neurons.

As described further herein, in some embodiments, the surrogate model is trained to predict the response of one or multiple sizes of neurons.

As described further herein, in some embodiments, the sequence of numerical input vectors is from a model of a simplified nerve morphology with an electrode.

As described further herein, in some embodiments, the surrogate model combines a cable surrogate that includes nodal compartments of a complete neuron model with a machine learning component and/or an artificial intelligence component.

Embodiments of the present disclosure include a method of administering neuromodulation therapy to a subject, the method comprising: programming a pulse generator to deliver a pattern of electrical stimulation identified using the method of for estimation of a neuronal response; and delivering the pattern of electrical simulation to the subject to generate a response in at least one target neuron.

### 4. Inverted Model of Neuronal Response

An optimization problem is solved to determine electrode geometry and/or stimulation parameters that achieve selective fiber activation in a model of neural stimulation. An optimization problem in this context is defined by a tissue morphology, consisting of a tissue geometry and tissue electrical conductivities, and the locations of neurons, with their own geometries, within that morphology. Along with these physical characteristics, the optimization problem is defined by labels that identify which neurons are targeted for stimulation (activation) or conduction block, and a loss function that grades the performance of a set of parameters with respect to achieving the desired selectivity, although the loss function may also incorporate other performance criteria as described below. The task is to then determine stimulation and/or electrode geometric parameters that selectively activate or block the targeted neurons by minimizing the associated loss function. Stimulation parameters may for example include the amplitudes, pulse widths, waveform shapes, and/or delays of current waveforms being delivered from each of several electrode contacts. Geometric parameters may for example include the inter-contact spacing for circumneural contacts in a cuff electrode, the number of such contacts, and/or the shape of such contacts. The loss function may be single or multi-objective, wherein the loss criteria may incorporate: minimizing energy required for selective activation or block, minimizing power required for selective activation or block, minimizing charge imbalance in the optimized waveform(s), minimizing onset response produced when the optimized waveform(s) are turned on, maximizing degree of conduction block, minimizing voltage required for activation or block with the optimized waveform(s), minimizing current required for activation or block with the optimized waveform(s), minimizing charge required for activation or block with the optimized waveform(s), maximizing therapeutic benefit produced by application of the optimized waveform(s), minimizing adverse effects produced by application of the optimized waveform(s), maximizing selectivity of activation or block between nerve fiber types by application of the optimized waveform(s), maximizing selectivity of activation or block between nerve fiber diameters by application of the optimized waveform(s), maximizing selectivity of activation or block between nerve fiber locations by application of the optimized waveform(s), measures related to the complexity of the electrode geometry or stimulation waveform shape, and any combinations and derivatives thereof.

In the single-objective case, an example loss function for maximizing, e.g., selectivity in activation, is the Jaccard loss:

$$\frac{\left|A ∪ B\right| - \left|A ∩ B\right|}{\left|A ∪ B\right|} ,$$

where A and B are ordered lists of 0′sand 1′s indicating whether each neuron did not fire or did fire an action potential, the union is calculated with an OR operation, the intersection is calculated with an AND operation, and |X| is the cardinality of set X (i.e., sum of the set). This loss measures the dissimilarity in overlap between two sets A and B. In the context of selective nerve stimulation, set A constitutes those neurons which are targeted for activation, and set B constitutes those which did in fact produce a propagating action potential (as predicted by a computational model, which may be a biophysical neuron model, or a surrogate model as described herein, or some other approximation of neuronal activity, such as an activation function or linear threshold estimator). The Jaccard loss has a theoretic minimum of 0, where sets A and B fully overlap (i.e., all targeted neurons are activated and no off-target neurons are activated), and a maximum of 1. Other loss functions for binary labeled data such as the Hamming loss, binary cross entropy loss, Hinge loss, etc., may also be used. Additional criteria as outlined above can be integrated in the single-objective context by applying a weighted sum over the various criteria. In the multi-objective case, the loss function returns an n-tuple of n objectives, where each of those objectives is computing some loss criterion as outlined above - for example, if integrating energy minimization as an objective, the loss function may return a 2-tuple consisting of the Jaccard loss to assess selectivity and the summed squares of all current waveforms emitted from all electrode contacts to assess electric power expenditure.

Method disclosed herein apply gradient-free and gradient-based approaches to solve optimization problems of this kind. In both cases, neuronal responses to stimulation may variously be computed using biophysical models (e.g., to validate solutions, validate responses at boundaries of regions of activation, block, or subthreshold response, predict all responses for a given set of stimulus parameters, and combinations and derivatives thereof) or surrogate models as described above; however, gradients may only be computed using an appropriate surrogate model. Activity metrics used when computing loss functions may consist of checking for a variable (e.g., transmembrane voltage(s) or current(s), or variables representing the state of conduction of individual ion channels) crossing some predetermined threshold or may consist of functions of some variables such as the mean value of a gating variable over some time window for some set of compartments, or any combinations and derivatives thereof.

With reference to FIG. 9, a high-level scheme for a gradient-free optimization is illustrated. In one implementation, an adapted Differential Evolution algorithm is used, although other gradient-free optimization methods such as genetic algorithms, particle swarm optimization, or simulated annealing may be used. At each iteration of the algorithm, the population is updated through a process of mutation, crossover, and selection. A ‘trial vector’ U_{i,G} is constructed for each member X_{i,G} of the population in iteration G following a mutation rule and crossover operation. As an illustration, EQN. 12 provides the greedy ‘best1’ mutation rule.

$${V}_{i , G} = {X}_{b e s t , G} + F ⋅ \left({X}_{r 0 , G} - {X}_{r 1 , G}\right) \qquad \text{[EQN. 12]}$$

where X_{best,G} is the best candidate solution in the current iteration, F is the mutation rate, X_{r0,G} and X_{r1,G} are mutually exclusive randomly selected candidate solutions from the current population. Most state-of-the-art metaheuristic optimization algorithms have mechanisms to adapt how they search a domain based on the topology of the loss function. The use of a differential mutation operator oriented around a difference vector (X_{r0,G} - X_{r1,G} in the ‘best1’ mutation rule illustrated above) achieves this by implicitly adapting the search range and direction to the loss function, a process referred to as ‘contour fitting’. U_{i,G} is then constructed from V_{i,G} via crossover: for each j ∈ 0, 1 ... d where d is the dimensionality of the optimization problem,

$${U}_{i , G}^{j} = {V}_{i , G}^{j} \text{if} r a n d \left(0 , 1\right) < \text{CR, else} {U}_{i , G}^{j} = {X}_{i , G}^{j} ,$$

where rand(0, 1) is a random sample from a uniform distribution in the interval [0, 1) (resampled for every j) and CR is the crossover rate. In some implementations, if the resulting trial vector U_{i,G} exceeds the problem bounds in any dimension, the mutation and crossover operation is repeated up to a limit of 10 times, after which those dimensions that remain outside the bounds are randomly reinitialized to values within the bounds. This is a feasibility-preserving strategy for boundary constraint handling that has shown advantages over Darwinian (i.e., where boundary violations are discouraged by adding a penalty term to the loss function) and repair (i.e., where dimensions in which the boundary has been violated are placed back within the bounds following some rule, such as reflection against the boundary) methods in recent empirical studies using DE.

For single-objective problems, selection means U_{i,G} replaces X_{i,G} in iteration G + 1 if f(U_{i,G}) < f(X_{i,G}), i.e., if the loss of U_{i,G} is less than the loss of X_{i,G}, or equivalently, U_{i,G} has a better performance. In the multi-objective case, U_{i,G} replaces X_{i,G} only if f(U_{i,G}) dominatesf(X_{i,G}), that is f(U_{i,G}) ≤ f(X_{i,G}) in all objectives and f(U_{i,G}) < f(X_{i,G}) in at least one objective. Conversely, if f(X_{i,G}) dominatesf (U_{i,G}), U_{i,G} is discarded. If f (U_{i,G}) does not dominate f (X_{i,G}) or vice versa, U_{i,G} is appended to the population. After all candidate vectors have been constructed, evaluated, and compared, if the population size exceeds the set population number, the population is sorted into pareto fronts (groups within which no member dominates any others and which are themselves ranked based on which fronts dominate others), and the population is reduced to the set population size by randomly discarding the required number of members from the lowest ranked fronts.

The values F and CR, the mutation and crossover rates, respectively, are hyperparameters that are supplied to the optimizer upon initiation. Additionally, the implementation employs parameter adaptation, allowing the optimizer to learn better F and CR values during optimization. Under parameter adaptation, at each iteration, instead of using the single value CR for each crossover operation when generating each trial vector, a crossover rate CRᵢ is generated independently for each member of the population, sampling from a normal distribution with mean CR and standard deviation of 0.1, and subsequently clipped to [0, 1]. Let S_{CR} be the set of all such crossover rates in the current iteration for which the resulting trial vector U_{i,G} successfully replaced the corresponding target vector X_{i,G} (i.e., had a lower loss / dominated the target vector). Then in the next iteration CR is updated such that CR = (1 - c) ∗ CR + c ∗ mean_{A}(S_{CR}), where mean_{A} is the arithmetic mean and c is a positive number between 0 and 1. Likewise, instead of using the single value F for each mutation operation when generating each trial vector, a mutation rate Fᵢ is generated independently for every member of the population by random sampling from a Cauchy distribution with location parameter F and scale parameter 0.1, truncated to 1 if Fᵢ > 1 and regenerated if Fᵢ ≤ 0. Using the same terminology as before, in the subsequent iteration F is updated such that F = (1 - c) ∗ F + c ∗ mean_{L}(S_{F}), where mean_{L}(S_{F}) is the Lehmer mean

$$= \frac{{\sum }_{F ∈ {S}_{F}} F \hat{} 2}{{\sum }_{F ∈ {S}_{F}} F} .$$

The implementation uses a value of c = 0.1.

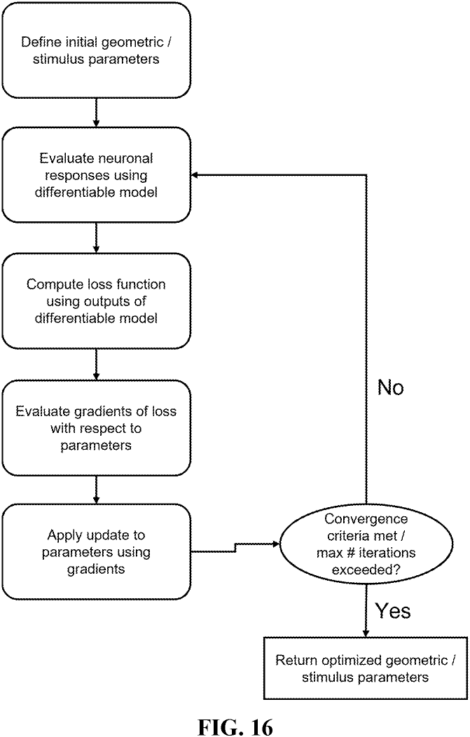

Gradient-based optimization: With reference to FIG. 16, a high-level scheme of gradient-based optimization for parameter selection is illustrated. Stochastic gradient-free algorithms such as Differential Evolution as described above may not perform well when the decision space is large, for example, when the class of parameterized stimulus to be used has not been defined (as might be the case if, for example, you wanted to minimize energy consumption in addition to maximizing selectivity and had no assumptions about the best waveform shape). Methods disclosed herein apply gradient descent to solve high-dimensional parameter selection problems in peripheral nerve, for example, stimulation using the differentiable dynamics models.

Gradient descent operates on scalar valued differentiable loss functions. In some implementations, the difference between the mean (calculated over the entire simulated period) m gating variable value in the 10 nodes at each end of each axon in the target and off-target populations is used. Additional criteria as outlined above (e.g., charge balance, energy minimization, power minimization, etc.) may be incorporated into the loss function through (weighted) addition. Let L be the scalar valued differentiable loss function that operates on the output of the differentiable dynamics model. Then, efficiently compute the derivatives of the loss with respect to the input parameters of the stimulation model, denoted ∇ₚL, where p is the vector of input parameters. p may consist of the parameters of various stimulus waveforms, or in cases where the waveform has not been predefined, it may consist of the numerical value of the stimulus current at every timestep for every electrode contact over the course of the simulation. If the model of the extracellular electric field is itself also differentiable (for example, in the case of point-source models in homogenous media), p may also contain parameters defining the size and or positions of extracellular sources of current. One update of the parameters in a single iteration of the standard gradient descent algorithm is then given by ← p - λ · ∇ₚL, where λ is the learning rate. In some implementation, an adaptive learning rate is used beginning at 0.01 and shrinking linearly to 0.001 over 500 iterations of gradient descent. However, variations on standard gradient descent, for example incorporating adaptive momentum with different learning rates and schedules, may be used. Additionally, gradient descent may be terminated when various convergence criteria are met, for example when the loss function has not decreased by a pre-specified amount for a pre-specified number of gradient descent iterations, or when magnitudes of the gradients of the loss function with respect to the optimizable parameters has dropped beneath a pre-specified value.

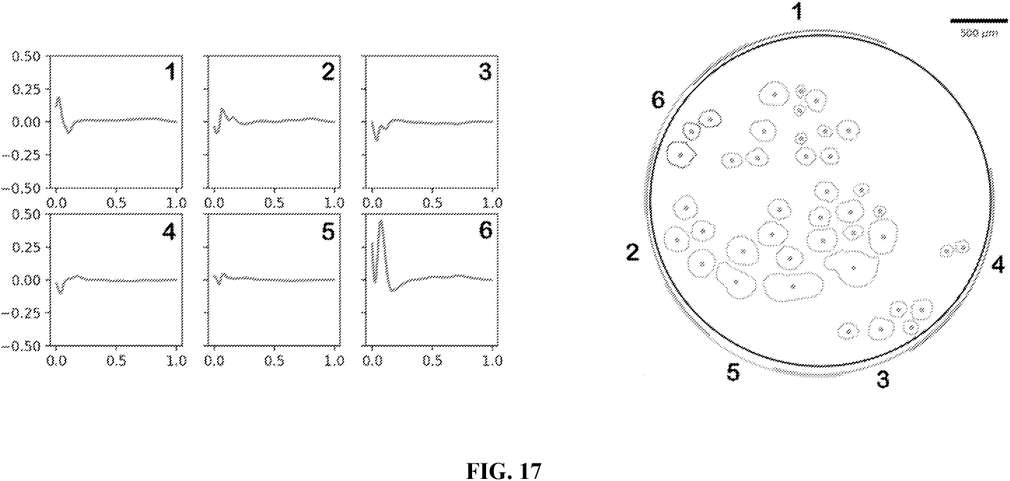

FIG. 17 shows the results of gradient descent in a pure selectivity task (i.e., the only loss criterion was target selectivity, without incorporating, for example, waveform charge balance) where the waveforms have not been specified. The output parameters (i.e., the model parameters being optimized) were the current amplitudes in mA at every timestep for each of 6 contacts in a cuff electrode for a 2.5 ms simulation with a timestep of 0.005 ms, totaling 3000 optimizable parameters. In the illustrated implementation, perfect selectivity is achieved.

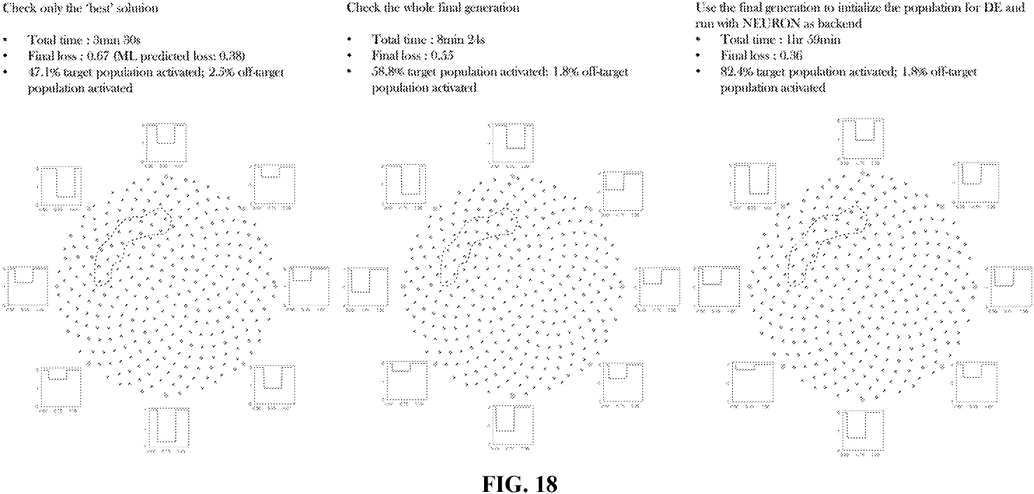

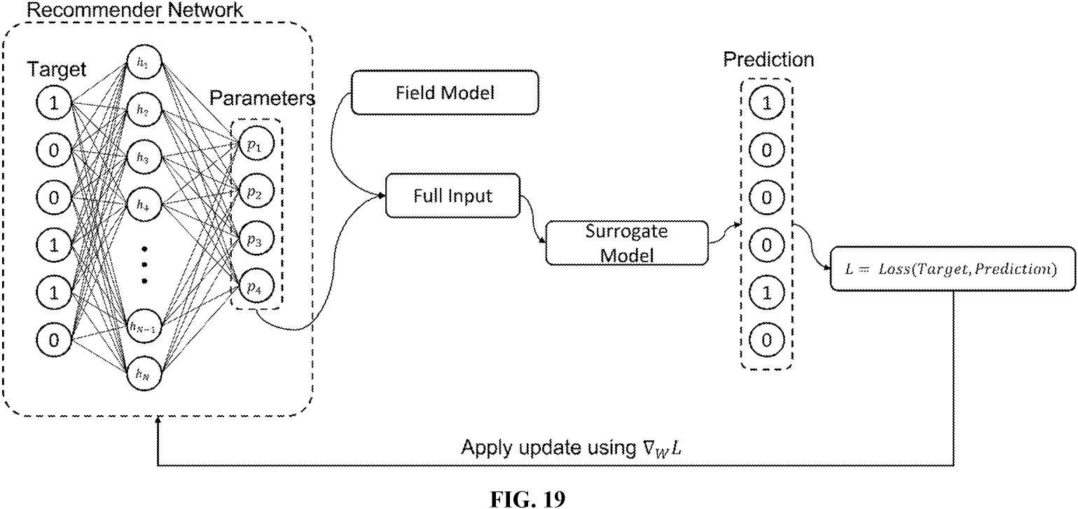

In both the gradient-free and gradient-based case, if using a surrogate model to evaluate performance, the results may be validated using biophysical neuron models. The results of these validations constitute additional extracellular electric field-neural response pairs that can be integrated into the training dataset and used to continue learning the models of the dynamics of activation and or block. Successful parameters may then be used to inform design of electrode geometries and/or stimulation parameters. Otherwise, the results of optimization using the learned surrogate models can be used to initialize the population for a gradient-free optimization using the biophysical neuron models to compute the loss function. This can overcome poor population initialization (whereby the gradient-free optimization method using the biophysical model to evaluate the loss function struggles to minimize the loss function due to low population diversity and no ‘good’ parameters in the population from which to construct superior candidates) and often converges to superior solutions compared to random initialization methods, as illustrated in FIG. 18.

Embodiments of the present disclosure include a method for optimizing neuronal response, the method includes inputting a set of criteria corresponding to a desired neuronal response into an inverted model of neuronal response. The inverted model of neuronal response outputs one or more stimulation criteria capable of producing the desired neuronal response.

As detailed herein, in some embodiments, the set of criteria corresponding to the desired neuronal response comprises identifying one or more target neuron(s) for activation or conduction block, and neurons having a specific subthreshold response.

As detailed herein, in some embodiments, the one or more stimulation criteria capable of producing the desired neuronal response is optimized with a gradient based optimization.

As detailed herein, in some embodiments, the one or more stimulation criteria capable of producing the desired neuronal response is optimized with a global optimization algorithm comprising at least one of: a differential evolution algorithm, a genetic algorithm, a particle swarm algorithm, a simulated annealing algorithm, an ant colony algorithm, an estimation of distribution algorithm, a simplex search algorithm, a coordinate descent algorithm, a random search algorithm, a grid search algorithm, a spiral optimization algorithm, a stochastic diffusion search algorithm, or any combination thereof.

As detailed herein, in some embodiments, the one or more stimulation criteria capable of producing the desired neuronal response is optimized for at least one of: minimizing energy required for activation or block or a subthreshold response, minimizing power required for activation or block or a subthreshold response, minimizing charge imbalance in the optimized waveform(s), minimizing onset response produced when the optimized waveform(s) are turned on, maximizing degree of conduction block, minimizing voltage required for activation or block or a subthreshold response with the optimized waveform(s), minimizing current required for activation or block or a subthreshold response with the optimized waveform(s), minimizing charge required for activation or block with the optimized waveform(s), maximizing therapeutic benefit produced by application of the optimized waveform(s), minimizing adverse effects produced by application of the optimized waveform(s), maximizing selectivity of activation or block between neuron types by application of the optimized waveform(s), maximizing selectivity of activation or block between neuron diameters by application of the optimized waveform(s), maximizing selectivity of activation or block between neuron locations by application of the optimized waveform(s), measures related to the complexity of the electrode geometry or stimulation waveform shape, and any combinations thereof.

As detailed herein, in some embodiments, the one or more stimulation criteria comprise an electrode geometric parameter comprising at least one of: inter-contact spacing of contacts, number of contacts, shape of contacts, size of contacts, shape of insulation, size of insulation, placement of contacts relative to the insulation, and any combination thereof.

As detailed herein, in some embodiments, the one or more stimulation criteria comprise an electrode placement parameter comprising at least one of: electrode implantation depth, electrode displacement from an anatomical landmark, rotation of electrode, and any combination thereof.

As detailed herein, in some embodiments, the one or more stimulation criteria comprise a stimulation parameter comprising at least one of: amplitude, pulse width, frequency, waveform shape, delays, and any combination thereof.

As detailed herein, in some embodiments, the method further includes training a recommender system with a gradient based optimization.

As detailed herein, in some embodiments, the one or more stimulation criteria from prior optimization(s) using a surrogate model is utilized to initialize subsequent optimization(s) using surrogate models(s) or complete biophysical model(s) to evaluate the neural response.

Embodiments of the present disclosure also include a method for estimation of one or more stimulation criteria. In accordance with these embodiments, the method includes inputting a sequence of numerical input vectors corresponding to a desired neuronal response to a surrogate model of neuronal stimulation. In some embodiments, the surrogate model outputs a sequence of numerical output vectors corresponding to one or more stimulation criteria capable of producing the desired neuronal response.

In some embodiments, the set of criteria corresponding to the desired neuronal response comprises identifying one or more target neuron(s) for activation or conduction block. In some embodiments, the set of criteria corresponding to the desired neuronal response comprises identifying one or more target neuron(s) having a specific subthreshold response.

In some embodiments, the one or more stimulation criteria capable of producing the desired neuronal response is optimized with a gradient based optimization.

In some embodiments, the one or more stimulation criteria capable of producing the desired neuronal response is optimized with a global optimization algorithm comprising at least one of: a differential evolution algorithm, a genetic algorithm, a particle swarm algorithm, a simulated annealing algorithm, an ant colony algorithm, an estimation of distribution algorithm, a simplex search algorithm, a coordinate descent algorithm, a random search algorithm, a grid search algorithm, a spiral optimization algorithm, a stochastic diffusion search algorithm, or any combination thereof; or optimized with a recommender system with a machine learning component and/or an artificial intelligence component comprising at least one of: recurrent neural network, convolutional neural network, fully connected neural network, decision tree, random forest, support vector machine, linear regression, logistic regression, nearest neighbors, or any combination thereof.

In some embodiments, the one or more stimulation criteria capable of producing the desired neuronal response is optimized for at least one of: minimizing energy required for activation or block or a subthreshold response, minimizing power required for activation or block or a subthreshold response, minimizing charge imbalance in the optimized waveform(s), minimizing onset response produced when the optimized waveform(s) are turned on, maximizing degree of conduction block, minimizing voltage required for activation or block or a subthreshold response with the optimized waveform(s), minimizing current required for activation or block or a subthreshold response with the optimized waveform(s), minimizing charge required for activation or block with the optimized waveform(s), maximizing therapeutic benefit produced by application of the optimized waveform(s), minimizing adverse effects produced by application of the optimized waveform(s), maximizing selectivity of activation or block between neuron types by application of the optimized waveform(s), maximizing selectivity of activation or block between neuron diameters by application of the optimized waveform(s), maximizing selectivity of activation or block between neuron locations by application of the optimized waveform(s), measures related to the complexity of the electrode geometry or stimulation waveform shape, and any combinations thereof.

In some embodiments, the one or more stimulation criteria comprise an electrode geometric parameter comprising at least one of: inter-contact spacing of contacts, number of contacts, shape of contacts, size of contacts, shape of insulation, size of insulation, placement of contacts relative to the insulation, and any combination thereof.

In some embodiments, the one or more stimulation criteria comprise an electrode placement parameter comprising at least one of: electrode implantation depth, electrode displacement from an anatomical landmark, rotation of electrode, and any combination thereof.

In some embodiments, the one or more stimulation criteria comprise a stimulation parameter comprising at least one of: amplitude, pulse width, frequency, waveform shape, delays, and any combination thereof.

Embodiments of the present disclosure include a method of administering neuromodulation therapy to a subject, the method comprising: programming a pulse generator to deliver a pattern of electrical stimulation identified using the method for optimizing neuronal response; and delivering the pattern of electrical simulation to the subject to generate a response in at least one target neuron.

### 5. Methods and Systems

The systems and methods described herein can be implemented in hardware, software, firmware, or combinations of hardware, software and/or firmware. In some examples, the systems and methods described in this specification may be implemented using a non-transitory computer readable medium storing computer executable instructions that when executed by one or more processors of a computer cause the computer to perform operations. Computer readable media suitable for implementing the systems and methods described in this specification include non-transitory computer-readable media, such as disk memory devices, chip memory devices, programmable logic devices, random access memory (RAM), read only memory (ROM), optical read/write memory, cache memory, magnetic read/write memory, flash memory, and application-specific integrated circuits. In addition, a computer readable medium that implements a system or method described in this specification may be located on a single device or computing platform or may be distributed across multiple devices or computing platforms.

One skilled in the art will readily appreciate that the present disclosure is well adapted to carry out the objects and obtain the ends and advantages mentioned, as well as those inherent therein. The present disclosure described herein are presently representative of preferred embodiments, are exemplary, and are not intended as limitations on the scope of the present disclosure. Changes therein and other uses will occur to those skilled in the art which are encompassed within the spirit of the present disclosure as defined by the scope of the claims.

### 6. EXAMPLES

It will be readily apparent to those skilled in the art that other suitable modifications and adaptations of the methods of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure, and should not be viewed as limiting to the scope of the disclosure.

The present disclosure has multiple aspects, illustrated by the following non-limiting examples.

### Example 1

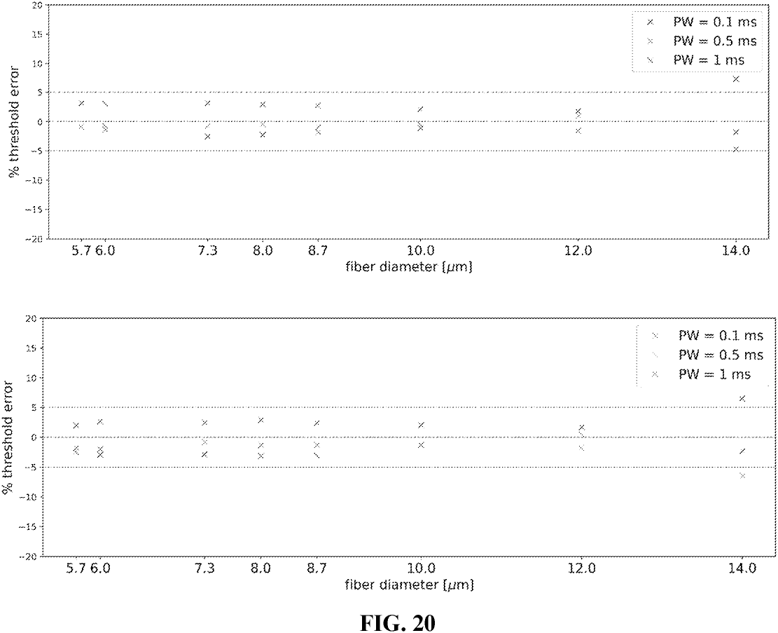

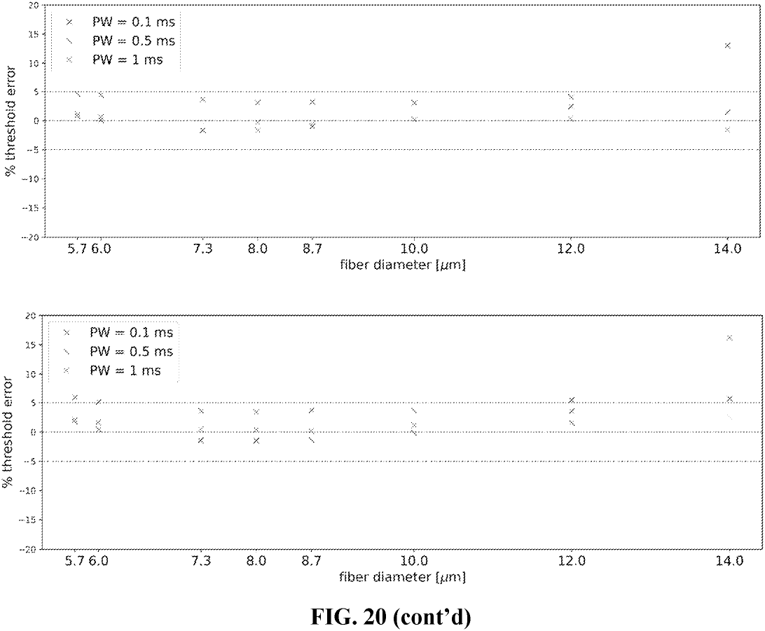

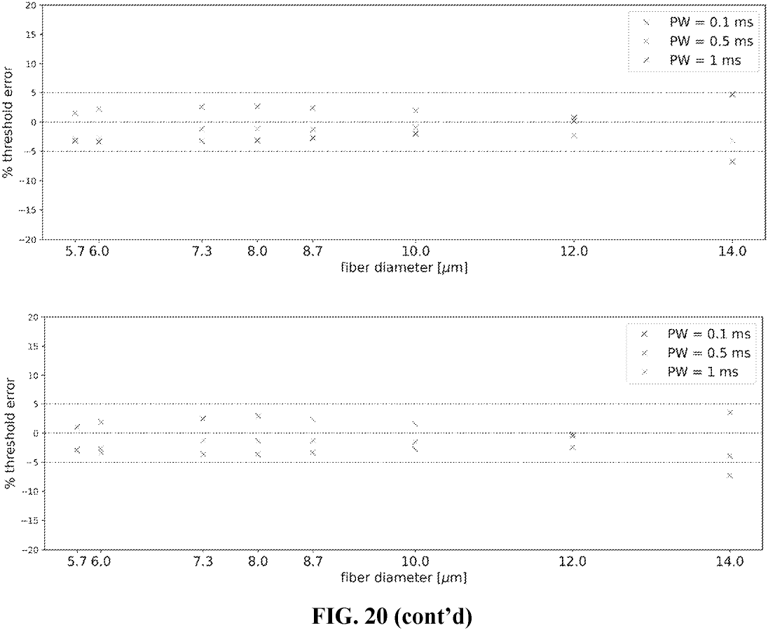

With reference to FIGS. 20-23, experiments were conducted to analyze the effects of fiber diameter and stimulus waveform on the cable surrogate prediction accuracy. For each tested waveform, single fibers of 5.7, 6, 7.3, 8, 8.7, 10, 12, and 14 µm diameter were placed in six random fascicles in a pig vagus nerve instrumented with a six-contact cuff; a different pig vagus nerve morphology was used in these simulations than used to generate data to train the cable surrogate. For each of the six fascicles in each nerve model, one of the six contacts from which to deliver stimulation was randomly selected and the surrogate predicted thresholds (T_{SURR}) were compared with NEURON predicted thresholds (T_{NRN}). Across all waveforms, fiber diameters and fascicles, a maximum absolute threshold error

$$\left|\left(100 \times \frac{{T}_{S U R R} - {T}_{N R N}}{{T}_{N R N}}\right)\right|$$

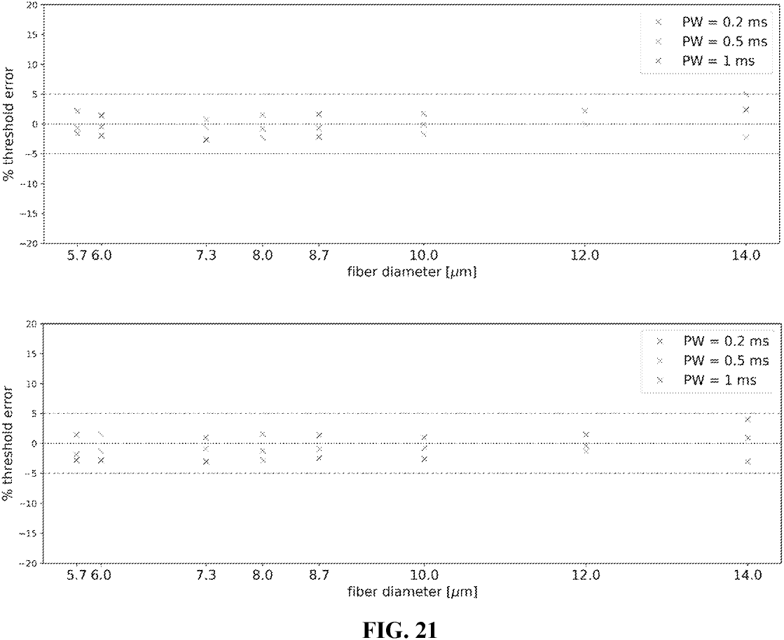

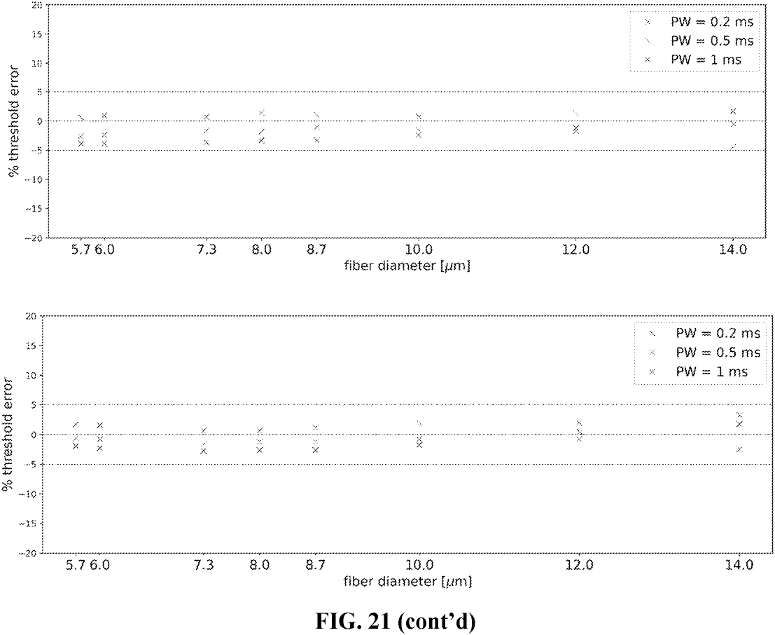

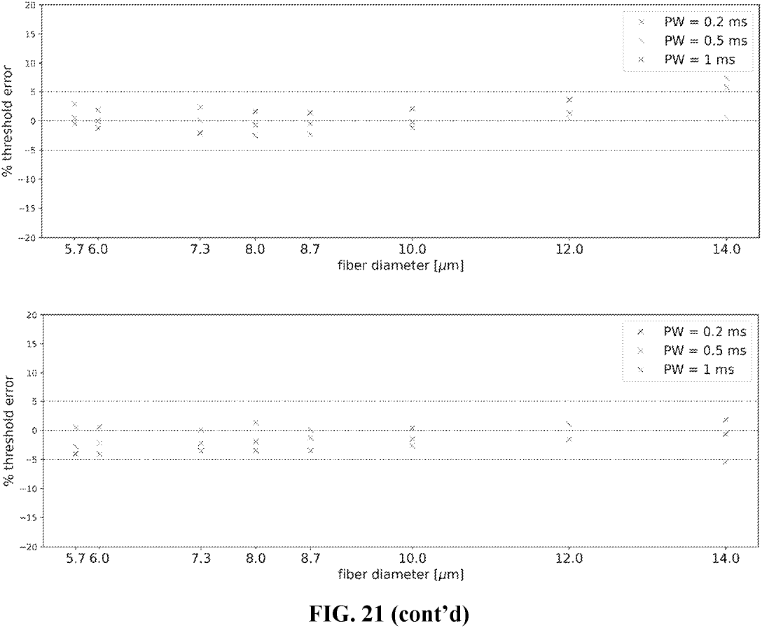

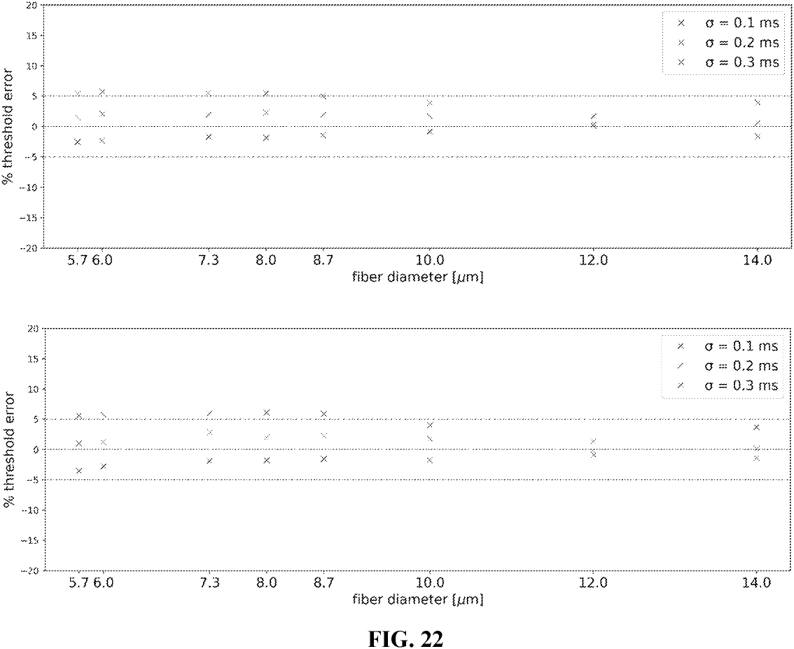

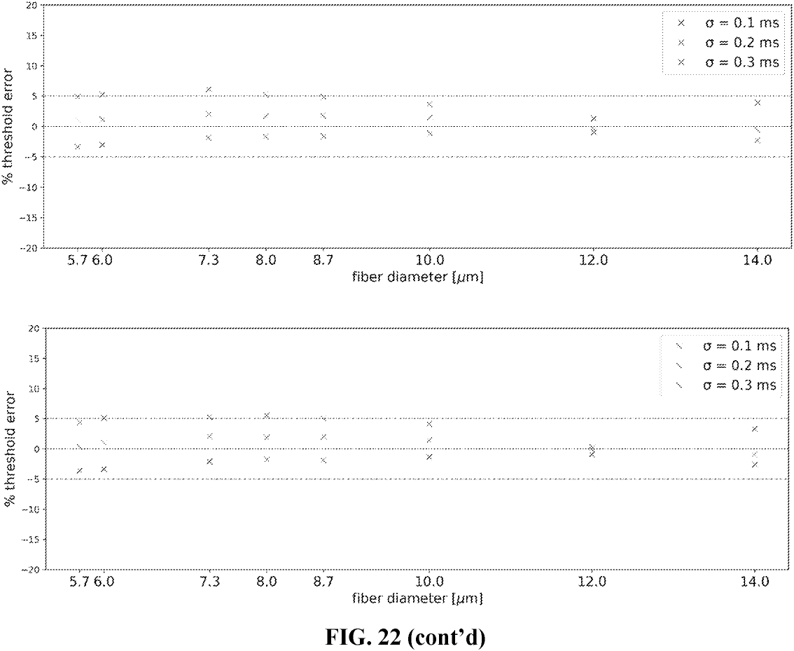

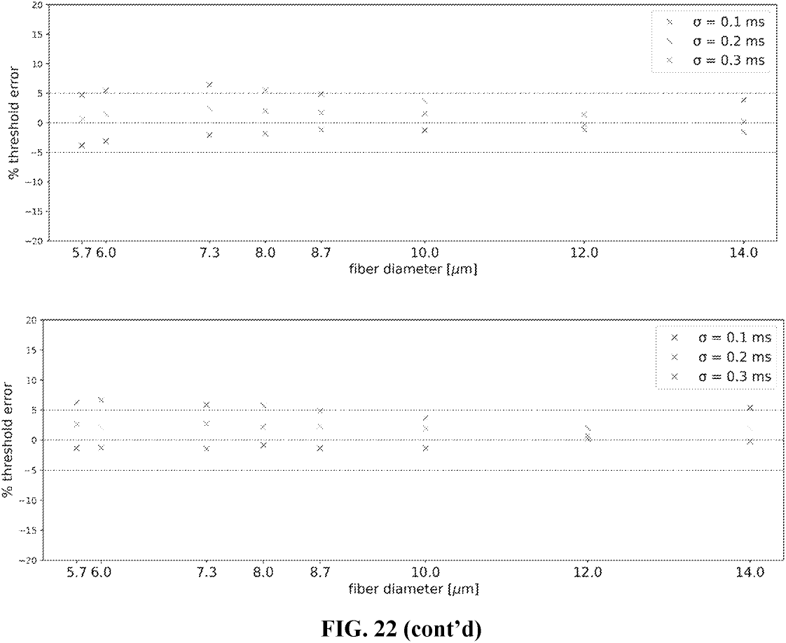

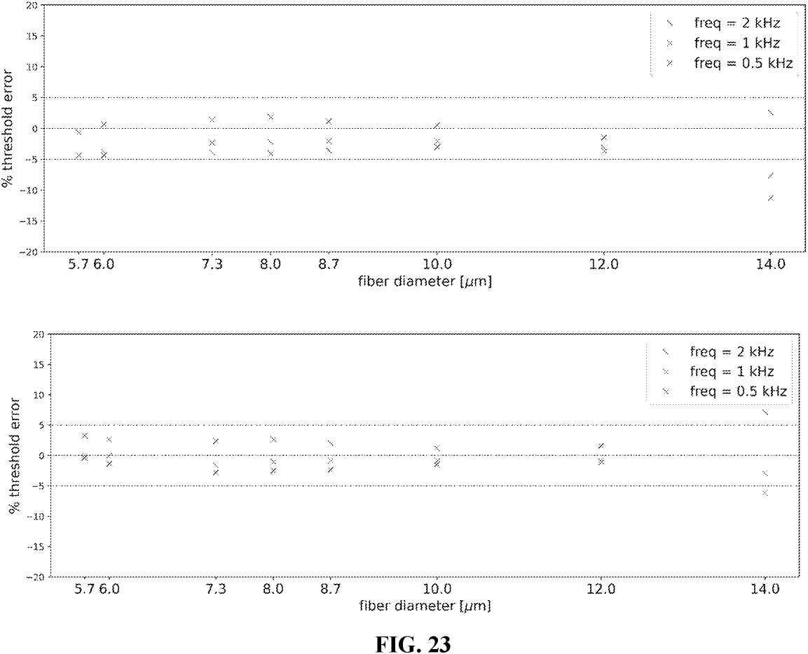

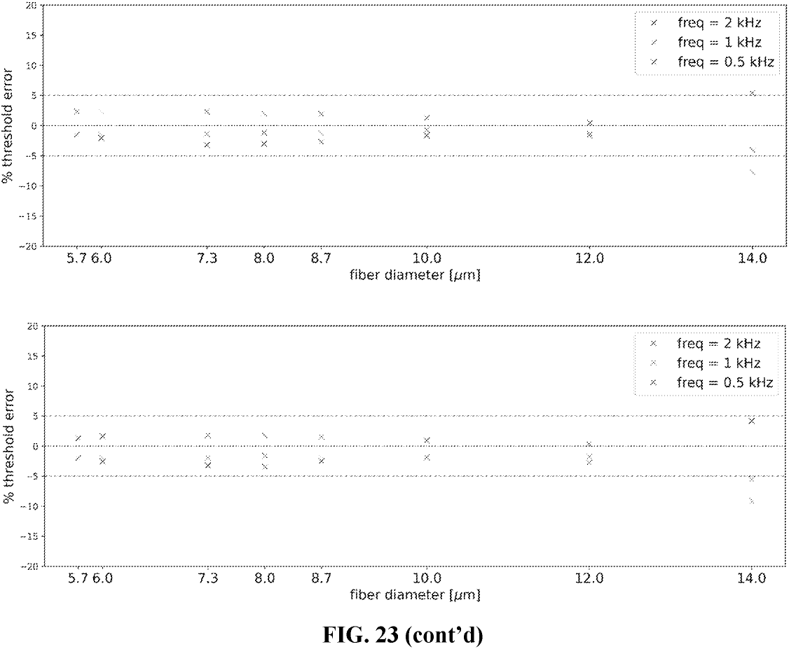

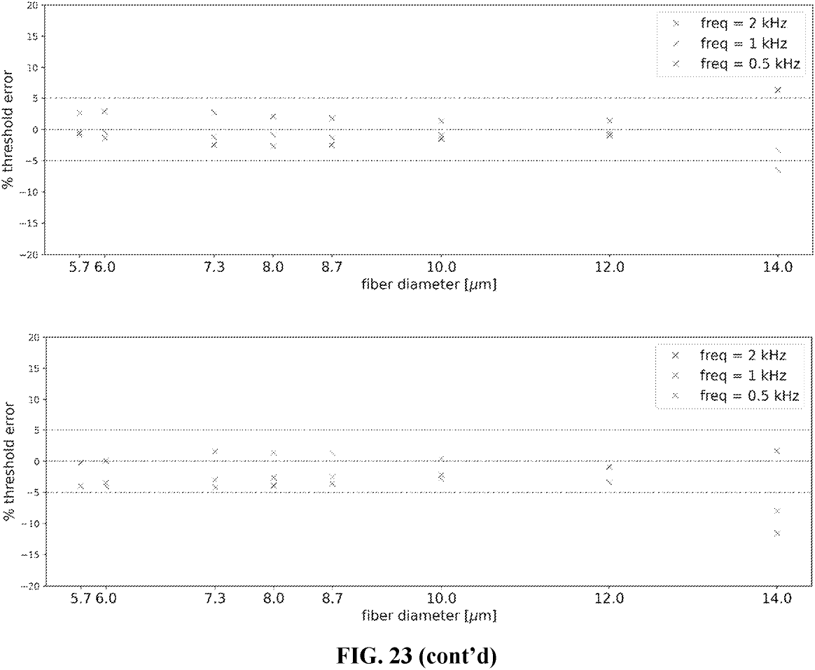

of approximately 15% (observed in the largest diameter fibers only) were observed. For nearly all diameters and waveforms, absolute threshold error was below 5%. It was found that fascicle position had relatively little effect on threshold error. Sawtooth (FIG. 20) exponential (FIG. 21), Gaussian (FIG. 22), and sinusoid (FIG. 23) waveforms were tested (i.e., all distinct from the class of waveform used to generate the training data), as shown below in Table 1.

TABLE 1

Definition of waveforms.

| | |
|---|---|
| Waveform | Mathematical Definition |
| Sawtooth | $\left(t - d e l a y\right) ∗ \frac{a m p}{p w} ∗ u \left(t - d e l a y\right) ∗ u \left(- t + d e l a y + p w\right)$ |
| Exponential | $a m p ∗ \left(\frac{{e}^{\frac{t}{τ}} - 1}{{e}^{\frac{p w}{τ}} - 1}\right) ∗ u \left(t - d e l a y\right) ∗ u \left(- t + d e l a y + p w\right)$ |
| Gaussian | $a m p ∗ {e}^{- \frac{1 {\left(t - μ\right)}^{2}}{2 {σ}^{2}}}$ |
| Sinusoid | $\begin{matrix}a m p ∗ s i n \left(2 ∗ π ∗ f r e q ∗ \left(t - d e l a y\right)\right) ∗ u \left(t - d e l a y\right) ∗ u \left(- t + d e l a y\right) \\ \left(+ \left(\frac{1}{f r e q}\right)\right)\end{matrix}$ |

### Example 2

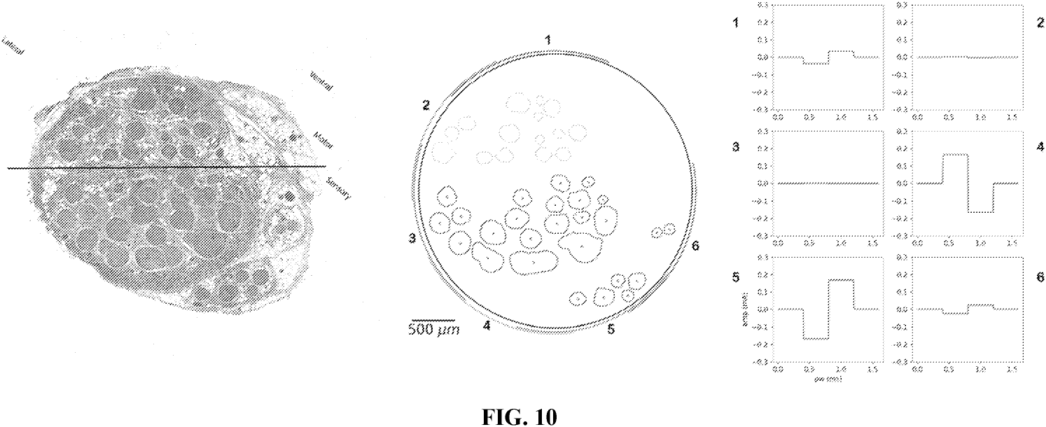

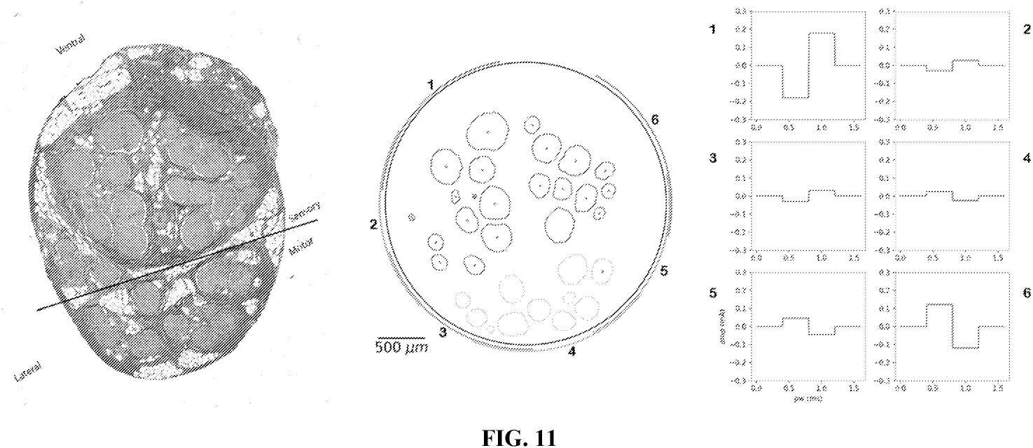

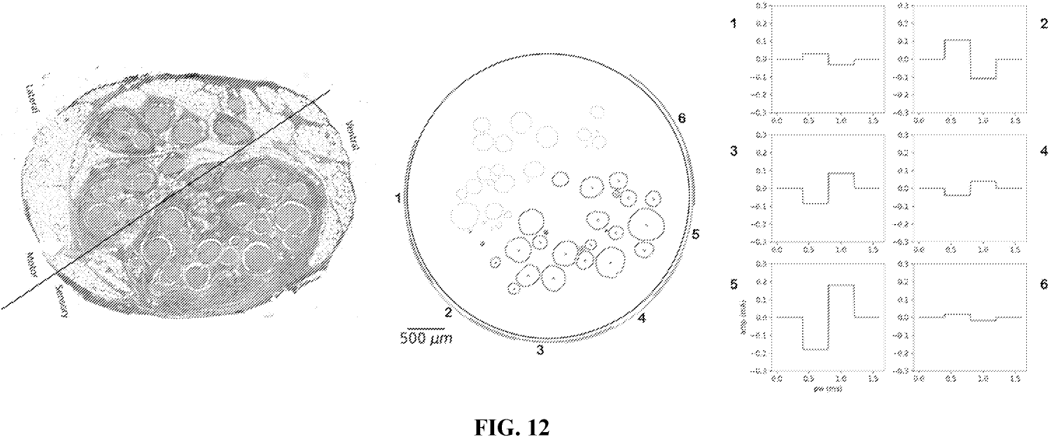

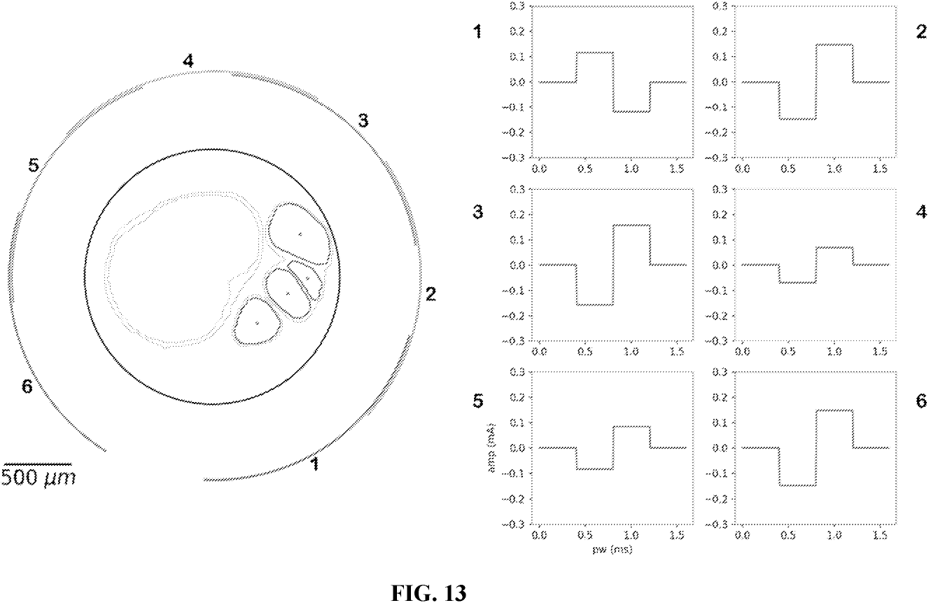

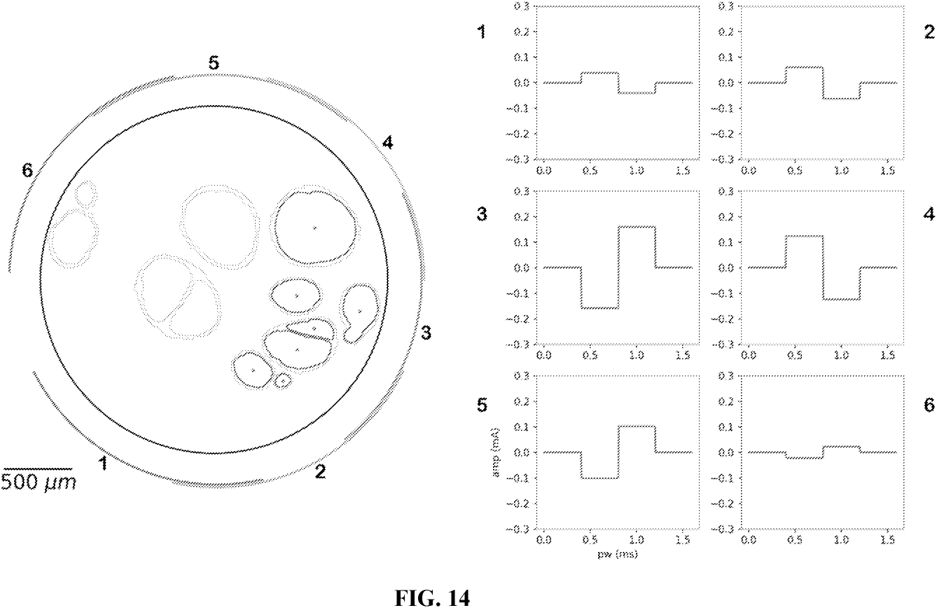

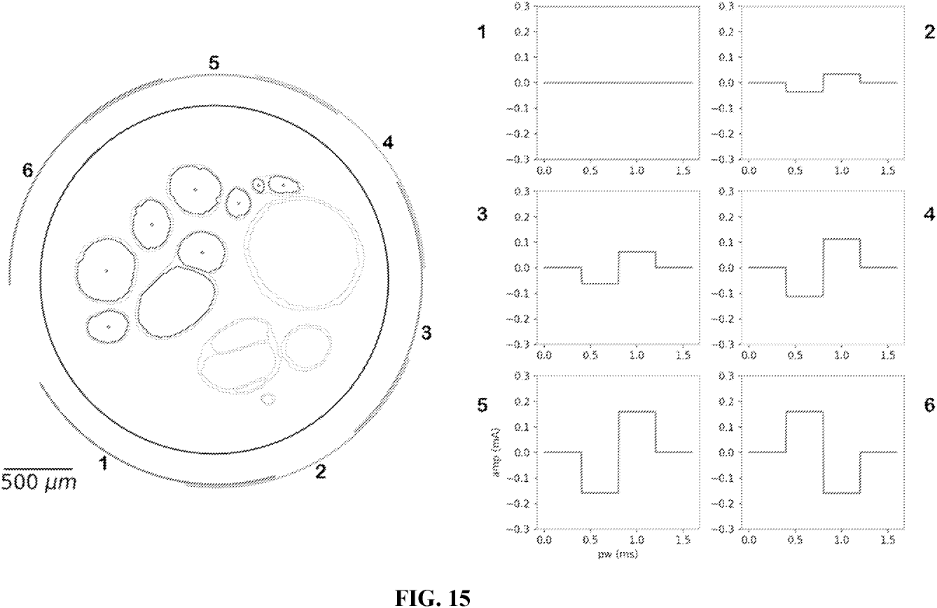

Experiments were also conducted to assess the selectivity performance using the gradient-free approach and the learned surrogate model (3-layer recurrent neural network trained to predict m, h, p, and s gating variables in the MRG axon model) in novel pig and human vagus nerve morphologies (FIGS. 10-15). The nerve was instrumented with a six-contact cuff electrode, and parameters optimized were stimulus amplitudes for each of the 0.4 ms pulse-width biphasic rectangular waveforms being delivered from each electrode contact. Each optimization took ~30 mins using a single GPU. The final optimization results are summarized in Table 2.

In general, where the predicted activation differs, the gating variable surrogate predicts a higher activation threshold.

TABLE 2

Final optimization performance

| | Predicted Performance (Surrogate Model) | | True Performance (NEURON) | |
|---|---|---|---|---|
| | Target activated (%) | Off-target activated (%) | Target activated (%) | Off-target activated (%) |
| Pig 1 | 100 | 0 | 100 | 0 |
| Pig 2 | 95.5 | 0 | 95.5 | 9.1 |
| Pig 3 | 88 | 0 | 92 | 4 |
| Human 1 | 100 | 0 | 100 | 0 |
| Human 2 | 100 | 0 | 100 | 0 |
| Human 3 | 88.9 | 0 | 88.9 | 0 |

### Example 3

Experiments were also conducted to assess gradient descent in a selectivity task where the waveforms have not been specified; the results of these experiments are provided in FIG. 17. The output parameters (i.e., the model parameters being optimized) were the current amplitudes in mA at every timestep for each of the 6 electrode contacts for a 2.5 ms simulation with a timestep of 0.005 ms for a total of 3000 optimizable parameters. The right-hand panel shows the cross section of a pig vagus nerve, where the inner shapes denote fascicles (bundles of axons), electrode contacts around the nerve are numbered 1-6; target fascicles are indicated in blue, with a single axon (5.7 µm MRG) placed at the centroid of each fascicle. Optimized waveforms for each of the contacts are on the left, with numbers corresponding to the electrode contact from which the waveform is delivered. The neuronal response as evaluated using the biophysical model (NEURON) is on the right - red dots indicate activated axons (i.e., axons that generated a propagating action potential), and green indicates axons that were note activated. As demonstrated, perfect selectivity was achieved (i.e., only axons in the target fascicles generated a propagating action potential).

It is understood that the foregoing detailed description and accompanying examples are merely illustrative and are not to be taken as limitations upon the scope of the disclosure, which is defined solely by the appended claims and their equivalents. Various changes and modifications to the disclosed embodiments will be apparent to those skilled in the art.

What is claimed is:

1. A method for estimation of a neuronal response, the method comprising:
inputting a sequence of numerical input vectors corresponding to an electric field over a period of neuronal stimulation into a surrogate model of neuronal stimulation;
wherein the surrogate model of neuronal stimulation outputs a sequence of numerical output vectors corresponding to one or more changes in neuronal response during the stimulation period.

2. The method of claim 1, wherein the sequence of numerical input vectors comprise a spatiotemporal distribution of extracellular potentials measured, calculated, or estimated for the period of neuronal stimulation.

3. The method of claim 1, wherein the one or more changes in neuronal response corresponding to the sequence of numerical output vectors comprise at least one of: transmembrane voltage, total transmembrane current, transmembrane current for one or more ions, conduction state of an ion channel, detection of one or more action potentials, a probability of a dynamic event, gating variable values, or any combination thereof.

4. The method of claim 1, wherein the surrogate model comprises a machine learning component and/or an artificial intelligence component comprising at least one of: recurrent neural network, convolutional neural network, fully connected neural network, decision tree, random forest, support vector machine, linear regression, logistic regression, nearest neighbors, or any combination thereof.

5. The method of claim 1, further comprising training the surrogate model of neuronal stimulation with data from simulation of a biophysical model or a simplified model.

6. The method of claim 1, wherein the sequence of numerical input vectors is from a model of an anatomically realistic nerve morphology with an electrode.

7. The method of claim 1, wherein the surrogate model is a classification surrogate that determines a probability that an action potential has occurred.

8. The method of claim 1, wherein the surrogate model determines a strength-duration relationship, determines a relationship between activation thresholds and neuron size, determines a relationship between block thresholds and neuron size, determines subthreshold response, identifies unidirectional propagation, identifies bidirectional propagation, determines propagation speed, determines the number of evoked action potentials, determines the time of action potential initiation, determines the location of action potential initiation, determines a gating variable, captures interaction between propagating action potentials, captures interaction between a subthreshold change in membrane state and a propagating action potential, captures interaction between changes in membrane state and responses to stimulation, predicts a state of conduction block, or any combination thereof.

9. The method of claim 1, wherein the surrogate model is a cable surrogate that includes nodal compartments of a complete neuron model.

10. The method of claim 1, wherein the surrogate model is fully differentiable with respect to all parameters.

11. A method of administering neuromodulation therapy to a subject, the method comprising:
programming a pulse generator to deliver a pattern of electrical stimulation identified using the method of claim 1; and
delivering the pattern of electrical simulation to the subject to generate a response in at least one target neuron.

12. A method for optimizing neuronal response, the method comprising:
inputting a set of criteria corresponding to a desired neuronal response into an inverted model of neuronal response;
wherein the inverted model of neuronal response outputs one or more stimulation criteria capable of producing the desired neuronal response.

13. The method of claim 12, wherein the set of criteria corresponding to the desired neuronal response comprises identifying one or more target neuron(s) for activation or conduction block.

14. The method of claim 12, wherein the one or more stimulation criteria capable of producing the desired neuronal response is optimized with a gradient based optimization.

15. The method of claim 12, wherein the one or more stimulation criteria capable of producing the desired neuronal response is optimized with a global optimization algorithm comprising at least one of: a differential evolution algorithm, a genetic algorithm, a particle swarm algorithm, a simulated annealing algorithm, an ant colony algorithm, an estimation of distribution algorithm, a simplex search algorithm, a coordinate descent algorithm, a random search algorithm, a grid search algorithm, a spiral optimization algorithm, a stochastic diffusion search algorithm, or any combination thereof.

16. The method of claim 12, wherein the one or more stimulation criteria capable of producing the desired neuronal response is optimized for at least one of: minimizing energy required for activation or block or a subthreshold response, minimizing power required for activation or block or a subthreshold response, minimizing charge imbalance in the optimized waveform(s), minimizing onset response produced when the optimized waveform(s) are turned on, maximizing degree of conduction block, minimizing voltage required for activation or block or a subthreshold response with the optimized waveform(s), minimizing current required for activation or block or a subthreshold response with the optimized waveform(s), minimizing charge required for activation or block with the optimized waveform(s), maximizing therapeutic benefit produced by application of the optimized waveform(s), minimizing adverse effects produced by application of the optimized waveform(s), maximizing selectivity of activation or block between neuron types by application of the optimized waveform(s), maximizing selectivity of activation or block between neuron diameters by application of the optimized waveform(s), maximizing selectivity of activation or block between neuron locations by application of the optimized waveform(s), measures related to the complexity of the electrode geometry or stimulation waveform shape, and any combinations thereof.

17. The method of claim 12, wherein the one or more stimulation criteria comprise an electrode geometric parameter comprising at least one of: inter-contact spacing of contacts, number of contacts, shape of contacts, size of contacts, shape of insulation, size of insulation, placement of contacts relative to the insulation, and any combination thereof.

18. The method of claim 12, wherein the one or more stimulation criteria comprise an electrode placement parameter comprising at least one of: electrode implantation depth, electrode displacement from an anatomical landmark, rotation of electrode, and any combination thereof.

19. The method of claim 12, wherein the one or more stimulation criteria comprise a stimulation parameter comprising at least one of: amplitude, pulse width, frequency, waveform shape, delays, and any combination thereof.

20. A method of administering neuromodulation therapy to a subject, the method comprising:
programming a pulse generator to deliver a pattern of electrical stimulation identified using the method of claim 12; and
delivering the pattern of electrical simulation to the subject to generate a response in at least one target neuron.
